(19) United States
(12) Patent Application Publication
(10) Pub. No.: US 2025/0325759 A1
(43) Pub. Date: Oct. 23, 2025

(54) INHALER DEVICE

(71) Applicant: Sandoz AG, Basel (CH)

(72) Inventors: James Anthony Tibbatts; Robin Craig Cocker; Cristian Popa; Paul Christopher Edward Mutti; Robert Borrett; Kesia Goodchild

(73) Assignee: Sandoz AG, Basel (CH)

(21) Appl. No.: 18/720,337

(22) PCT Filed: Dec. 13, 2022
(86) PCT No.: PCT/EP2022/085539

§ 371 Date: Jun. 14, 2024

(30) Foreign Application Priority Data
Dec. 17, 2021 (GB) 2118384.3

(51) Int. Cl. A61M 15/00

(52) U.S. Cl. CPC A61M 15/0003; A61M 2205/3331; A61M 2205/3379; A61M 2206/10; A61M 2210/0625

(57) ABSTRACT

The present invention relates to an inhaler device for use with an elongate strip medicament carrier comprising a base with a plurality of medicament pockets or blisters and a peelable lidding sheet covering the blisters, the inhaler device comprising; an indexer movable in a first direction to advance a blister pocket into an inhalation position; an opening system for peeling the peelable lidding sheet from the base to expose medicament within each medicament pocket; the opening system comprising a peel management component movable in a first direction corresponding to said first direction of the indexer to delay the peeling action when the indexer is advanced, and in a second, opposite, direction to allow the lidding sheet to be peeled from the base; wherein the indexer and peel management component are separately driven in said first direction so that movement of the indexer does not dictate movement of the peel management component.

The present disclosure relates to inhaler devices, in particular to devices provided with medicament carriers containing individual pockets or blisters of powder medicament covered by a lidding sheet.

### BACKGROUND OF THE INVENTION

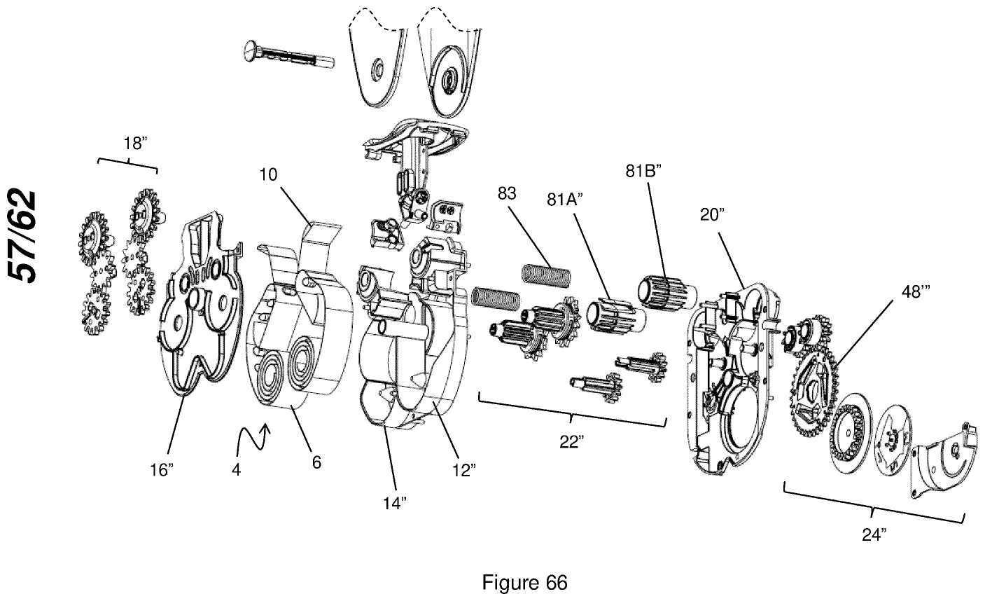

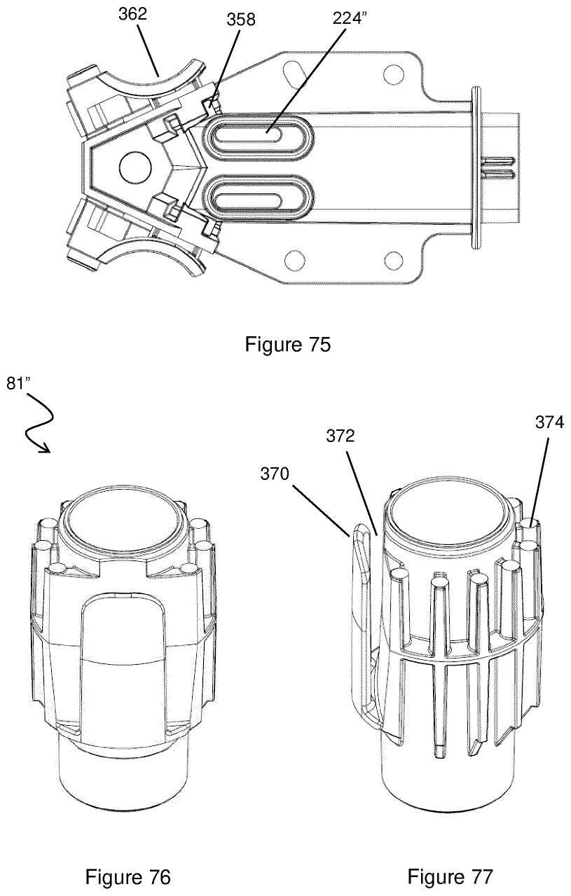

A prior art inhaler device is described in WO 2020/025977. Referring to FIGS. 66-79 of that application, the inhaler device comprises a moving airway 708. The airway 708 is operatively connected to a mouthpiece cover 710. The mouthpiece cover 710 is moveable to actuate a dispensing cycle of the inhaler. The moving airway 708 is moved as part of the dispensing cycle.

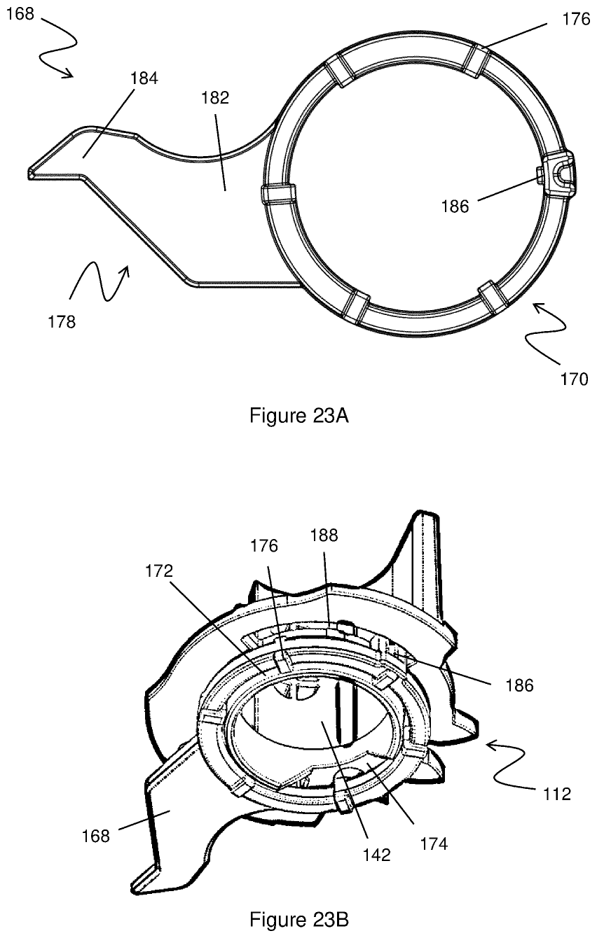

As most easily seen in FIGS. 23 and 23A, the airway 708 is placed adjacent the point where the lidding foil 170 is peeled back from a dose chamber 182. Thus, the foil 170 is peeled back, the medicament flows into the airway 170 via an aperture 16. The user may therefore inhale the medicament via the airway. Control of the peeling of the lidding foil is provided by peel beaks 790A,790A′. The peel beaks 790A, 790A′ are mounted to respective gears 790,790′ to provide movement thereof. The moving airway 708 engages the gears 790,790′ to effect movement of the gears 790,790′ (see FIGS. 70-77).

A space must be provided between the dose chamber 182 and the airway 708 to accommodate movement of the peel beaks 790A,790A′, and peeling of the lidding foil 170. In use, medicament may flow into the space rather than into the airway. This leads to an unpredictable dose of medicament and may leave residue etc. in the device, which may impair the function thereof.

According to the present invention there is provided an inhaler device as defined in the appended claim 1. Further optional features are recited in the associated dependent claims.

The inhaler device is designed for use with an elongate strip medicament carrier comprising a base with a plurality of medicament pockets or blisters and a peelable lidding sheet covering the blisters. It comprises an indexer movable in a first direction to advance a blister pocket into an inhalation position and an opening system for peeling the peelable lidding sheet from the base to expose medicament within each medicament pocket. The opening system comprises a peel management component which is movable in a first direction corresponding to said first direction of the indexer to delay the peeling action when the indexing system is advanced and in a second, opposite, direction to allow the lidding sheet to be peeled from the base. The indexer and peel management component are separately driven in said first direction so that movement of the indexer does not dictate movement of the peel management component.

There are benefits to advancing the indexer immediately on actuation. For example, the input force that needs to be applied to an actuator can be reduced by spreading the indexing operation across as much of the range of movement as possible. In previous devices an indexer and peel beak component are commonly driven, or one directly drives the other. In this known design, providing gearing that immediately engages to drive the indexer creates clash or interference problems with the input when the peel beak moves in first and/or second directions. As a result, the indexing or peel management is compromised. By separating driving of the peel management component and indexer, the problem does not arise.

Movement of the peel management component in the second direction may be at least partly effected by stored energy in the system.

The inhaler device may further comprise a drive feature for driving the peel management component in said first direction from a first position to a second position, and a release feature for releasing the peel management component from the second position.

Movement of the peel management component in the second direction may be from the second position to the first position. Said movement of the peel management component from the second position to the first position may be unimpeded, such that the peel management component is free to move back to the first position once released. The peel management component may engage with a drive feature in the first position. The drive feature may allow the peel management component to be moved back to the second position in a subsequent actuation.

The release feature and the drive feature may be provided on different layers of a common component, for example a gear mechanism or a component thereof such as a gear wheel.

The stored energy may be provided by a spring or by some other form of resilient member.

Movement of the peel management component in said first direction may be delayed relative to movement of the indexer in said first direction. The indexer may thus begin to move before the peel management component during use of the inhaler device

The peel management component and the indexer may be driven by two different parts of a common element, for example by two different layers of a common gear wheel or similar.

The inhaler device may comprise an input drive gear and one or more pawls, engaged with the input drive gear to allow rotation of the input drive gear in a first direction and to prevent rotation in a second direction, wherein each pawl acts on a substantially rotationally continuous surface of the input drive gear.

The inhaler device may comprise an input drive gear with a varying pitch radius providing a varying gear ratio with an engaged driven gear. Said input drive gear may have a repeated set profile of changing pitch radius around its circumference.

The set profile may be repeated multiple times, for example three or four times, around the circumference of the driving gear.

The inhaler device may comprise a unidirectional gear train in which one or more engaging gears comprise asymmetric gear teeth. The asymmetric teeth may be designed or configured to provide a preferable performance in one direction. Typical compromises in tooth design that are necessary to allow consistent performance in opposite drive directions can be avoided when a mechanism needs only to provide drive in one direction.

The invention also provides an inhaler device as defined in the appended claim 17. Further optional features are recited in the associated dependent claims.

The inhaler device is designed for use with a medicament carrier comprising a plurality of medicament pockets, and comprises an indexing system for advancing a medicament pocket into an inhalation position, an airway manifold for delivering medicament from a medicament pocket to a mouthpiece and a bridging element movable, perhaps about a pivot point, into engagement with an inlet to the manifold to bridge a gap between an exposed dose of a medicament pocket advanced to said inhalation position and said inlet to the manifold.

The bridging element allows spacing to be maintained between inhaler components, while still ensuring a complete, enclosed, path for medicament to the mouthpiece when required.

The bridging element may be movable or rotatable about a pivot point. The bridging element pivoting or swinging into engagement introduces less friction into the system, when moving into a position, than a sliding or otherwise moving bridging element.

The bridging element may comprise a sealing element for at least partly surrounding the inlet to the airway manifold to help maintain a sealed path or passageway from a medicament pocket to a mouthpiece of the device.

The medicament carrier may comprise a base with a plurality of medicament pockets and a peelable lidding sheet covering the pockets, and the inhaler device may comprise an opening system for peeling the peelable lidding sheet from the medicament carrier to open a blister.

The opening system may further comprise a movable peel beak component for regulating the peeling of a lidding sheet from a medicament carrier, wherein the peel beak component moves during advancement of the medicament carrier to said inhalation position, and wherein the bridging element extends from the peel beak component.

The indexing system may comprise a rotating indexer to advance a blister pocket from a first position into said inhalation position. The peel beak component May rotate with the indexer.

The airway manifold may comprise a recess for receiving and at least partly surrounding a free end of the bridging element.

The inhaler device may comprise first and second indexers for advancing first and second distinct medicament carriers. The first and second medicament carriers may be as described above, and the inhaler device may comprise an opening system per indexer for peeling the peelable lidding sheet from each medicament carrier to open respective blisters. A bridging element as described above may be provided for each of the first and second indexers.

Also provided is an inhaler device as defined in the appended claim 26. Further optional features are recited in the associated dependent claims.

The inhaler device is designed for use with one or more elongate strip medicament carriers comprising a base with a plurality of medicament pockets or blisters and a peelable lidding sheet covering the blisters. It provides first and second indexers, each movable in a first direction to advance a blister pocket into an inhalation position, and first and second opening systems for peeling the peelable lidding sheet from the base of a medicament carrier to expose medicament within each medicament pocket. The first opening system comprises a first peel management component movable in a first direction corresponding to said first direction of the first indexer to delay the peeling action when the first indexer is advanced, and in a second, opposite, direction to allow the lidding sheet to be peeled from the base. The second opening system comprising a second peel management component movable in a first direction corresponding to said first direction of the second indexer to delay the peeling action when the second indexer is advanced, and in a second, opposite, direction to allow the lidding sheet to be peeled from the base. The first and second peel management components interact such that the first peel management component cannot move from a position in which a medicament pocket is fully covered by the lidding sheet to a position in which the medicament pocket is fully uncovered by the lidding sheet without the second peel management component also moving from a position in which a medicament pocket is fully covered by the lidding sheet to a position in which the medicament pocket is fully uncovered by the lidding sheet.

The first and second indexers, opening systems and peel management components allow the inhaler device to peel open blisters on two different elongate strip medicament carriers, or from two ends different ends of a single elongate carrier. The two carriers or two ends may contain the same medicament or may contain different medicaments intended for inhalation together. Coordinating the movement of the first and second peel management components helps to ensure that neither the first nor second carrier/end has a blister opened and its medicament exposed before both are ready for inhalation, even if the first and second indexers are not advanced simultaneously.

The interaction between the first and second peel management components may comprise a system in which a first peel management component prevents substantial movement of a second peel management component until the first peel management component has substantially moved.

The inhaler device may further comprise a first lock for the first peel management component and a second lock for the second peel management component such that the first and second peel management components are independently lockable in respective positions in which respective medicament pockets are fully covered by respective lidding sheets.

The second peel management component may be released by the second lock before the first peel management component is released by the first lock.

The interaction between the first and second peel management components may comprise an interlock component

The interlock component may move rotationally on substantially the same axis as the first peel management component.

The invention also provides an airway manifold for inhaler device as defined in the appended claim 32. Further optional features are recited in the associated dependent claims.

The manifold comprises first and second manifold inlets for receiving first and second medicaments and a single outlet, and provides respective first and second fluid paths for medicament from the first and second manifold inlets to the single outlet. The manifold is designed or configured to provide a difference in flow through the first and second flow paths.

The difference in flow may be achieved in various different ways. In one example, the manifold may be at least partly divided into a first chamber fluidly connected to the first inlet and a second chamber fluidly connected with the second inlet. The first and second chambers may be asymmetrical/different in size and/or shape and/or design. The asymmetrical effect may alternatively be achieved without a central wall dividing the manifold into a first and second chamber; instead the medicament inlets and/or the bypass inlets may have asymmetrical sizes/angles/shapes/resistances, or the internal form of the manifold chamber may be asymmetrical, allowing differential effects to be applied to the drug delivery of the medicaments entering via different inlets.

Providing two different/asymmetric flow paths makes the manifold more versatile. Different formulations require different conditions for optimum Aerosol Particle Size Distribution (APSD), so when delivering two medicament formulations from a single device typically means that at least one is delivered under sub-optimal conditions. In the present invention, the size and/or geometry of each flow path can be separately configured or tuned to suit a particular formulation, so that each medicament passes through a space which provides tailored conditions for each of the medicaments to be delivered by the device. This helps to ensure the necessary APSD for each different medicament, with little or no need for compromise.

The first and second flow paths may be asymmetrical. For example, the volume of the first flow path may be different from the volume of the second flow path and/or the internal form of the first flow path may be different from the internal form of the second flow path and/or the surface finish of the first flow path may be different from the surface finish of the second flow path.

The manifold may be at least partly split into first and second chambers by a dividing wall, with the first chamber providing the first flow path and the second chamber providing the second flow path. Three or more chambers may be provided if desired, for example to provide three or more flow paths.

A separate bypass air inlet may be provided into each chamber. The bypass air inlets may be asymmetrical.

The first and second manifold inlets may be asymmetrical. For example, the size of the first manifold inlet may be different from the size of the second manifold inlet and/or the angle of the first manifold inlet may be different from the angle of the second manifold inlet and/or the surface finish of the first manifold inlet may be different from the surface finish of the second manifold inlet.

Alternatively, or additionally, an airway manifold may comprise a main manifold chamber and first and second passageways extending from the main manifold chamber and terminating at first and second inlets to receive entrained medicament, wherein one or more bypass flow channels are provided in the first and/or second passageways to allow flow from outside the manifold to join the entrained medicament flow.

The main manifold chamber may form an airway in communication with a mouthpiece, and the one or more bypass flow channels may be provided at or adjacent an inlet to the airway, Preferably the one or more bypass flow channels are provided so that the additional bypass flow is aligned with the entrained medicament flow. The flow may form a ‘sleeve’ of airflow to prevent medicament from impacting on and adhering to the internal surfaces of the manifold during use.

For example, a plurality of bypass flow channels may be provided around the periphery of the first and/or second passageways.

Separate bypass channels or apertures can thus be provided in separate passageways to provide bypass flow before the flows from first and second inlets join in a single main manifold chamber. This allows a separate/dedicated bypass flow to be associated with each of two different medicament sources. If appropriate, the bypass flow channels in the first and/or second passageways may be configured to provide asymmetric bypass flow in the first and second passageways. For example, any of the size, number, geometry, or surface finish of the bypass flow channels may differ between the first and second passageways. This may be beneficial in optimising the manifold for use with two different medicaments having different characteristics.

The main manifold chamber may be at least partially divided to provide first and second chambers as described above, and/or the manifold may comprise any of the additional features previously described.

The described airway manifold may from part of an inhaler device as previously described.

The invention also provides a resiliently deformable pawl as defined in the appended claim 46. Further optional features are recited in the associated dependent claims.

The pawl comprises first and second resilient limbs which are arranged at an angle to each other so that when the pawl is deformed as it moves past/over a ratchet tooth in use, one resilient limb is placed predominantly in compression rather than in bending and/or tension. It has been found that various materials, in particular certain plastic materials, experience less mechanical creep over time when subjected to compression than when subjected to comparable tension. Providing a resiliently deformable pawl with at least one limb that is at least predominantly compressed during deformation helps to prolong the useful life of the pawl and any associated components of a mechanism.

The angle between the first and second limbs at said vertex may be less than 90°, for example less than 30°, to help ensure that one limb is predominantly under compression rather tension during use. In one example the angle between the first and second limbs at said vertex is substantially 14°, more specifically 14.2°.

The pawl may be formed from a plastics material.

The pawl may be moulded as part of a gear wheel, and the gear wheel may form part of an inhaler device. Therefore, there is also provided is a gear wheel comprising a resiliently deformable pawl as defined above, and an inhaler device comprising such a gear wheel.

The pawl may be formed integrally with said gear wheel.

Examples of specific features of the invention will now be described with reference to the following drawings, in which:

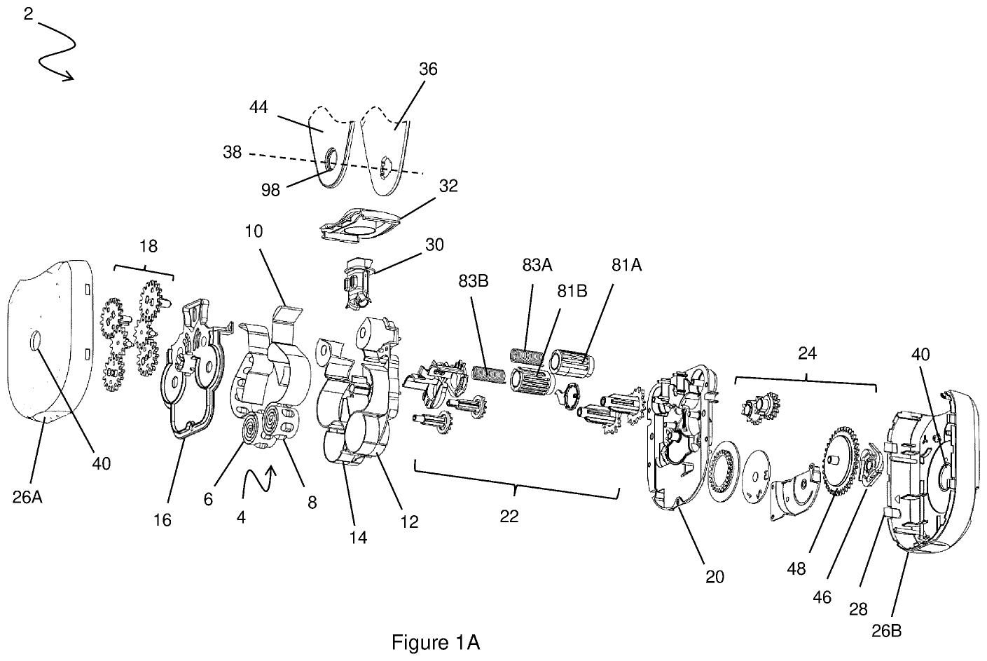

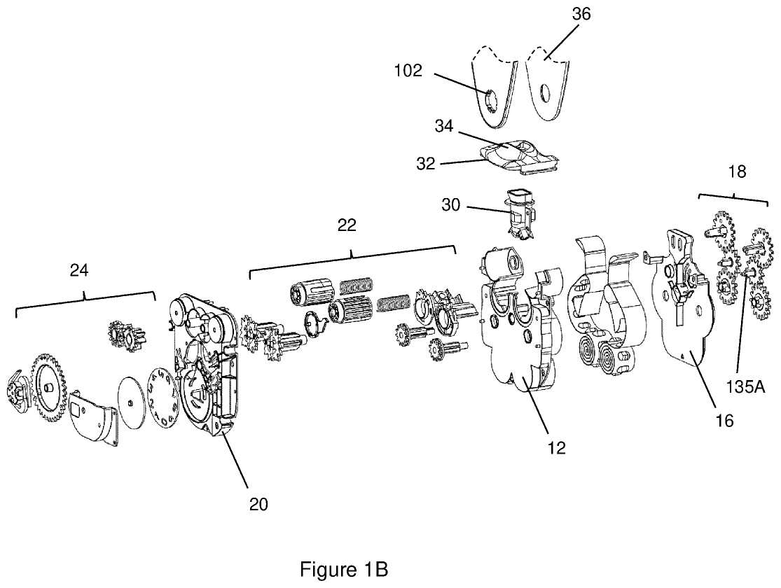

FIGS. 1A and 1B show exploded views of a first example inhaler device;

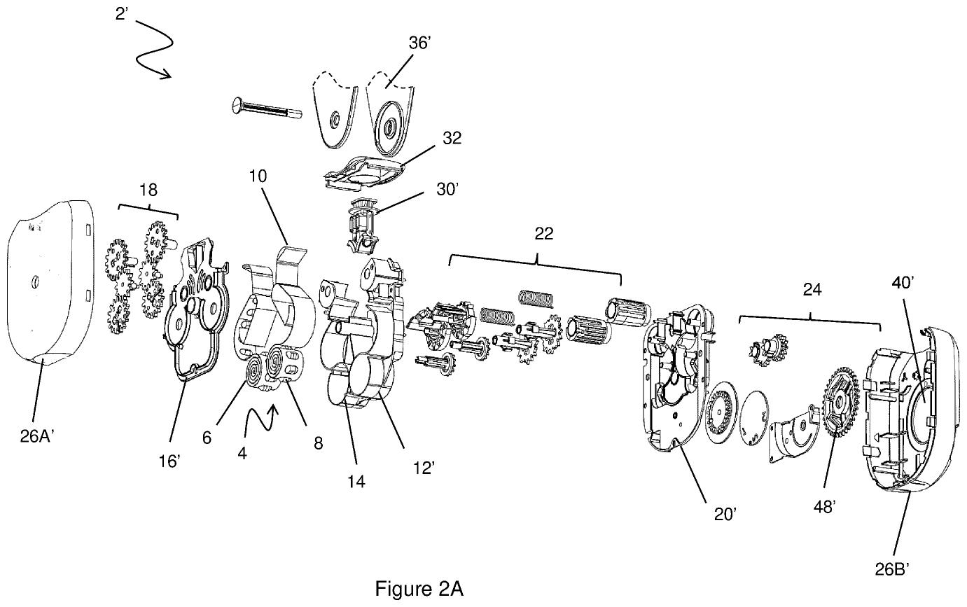

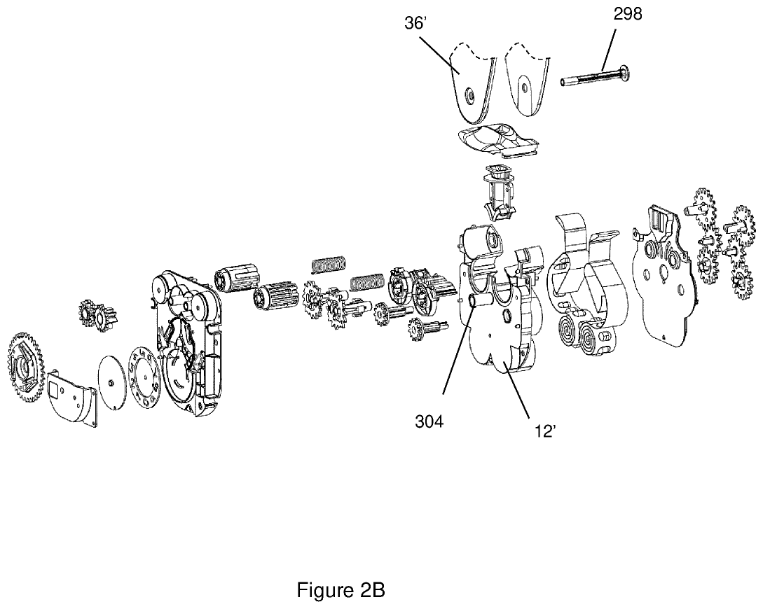

FIGS. 2A and 2B show exploded views of a second example inhaler device;

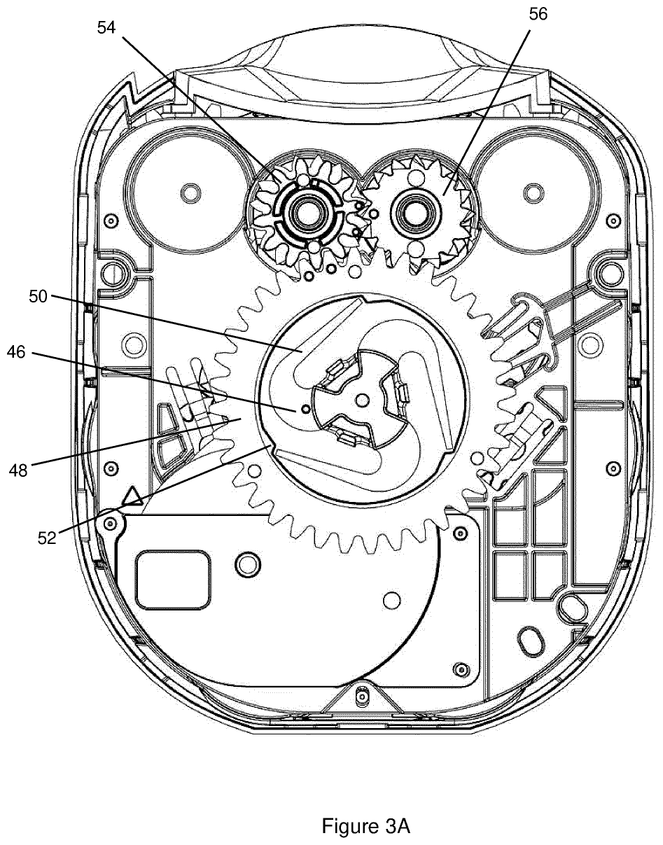

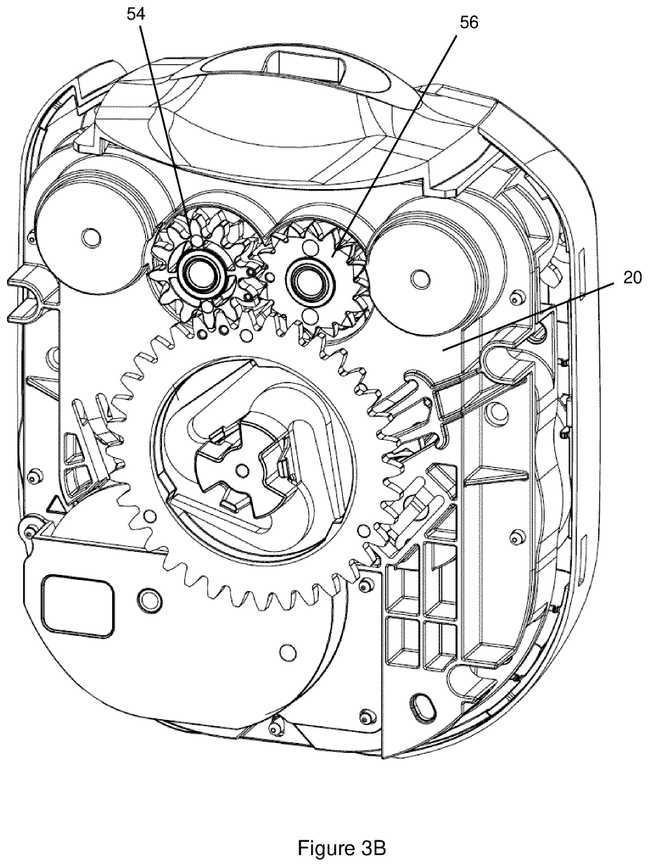

FIGS. 3A and 3B show a first part of the mechanism from the device of FIGS. 1A and 1B;

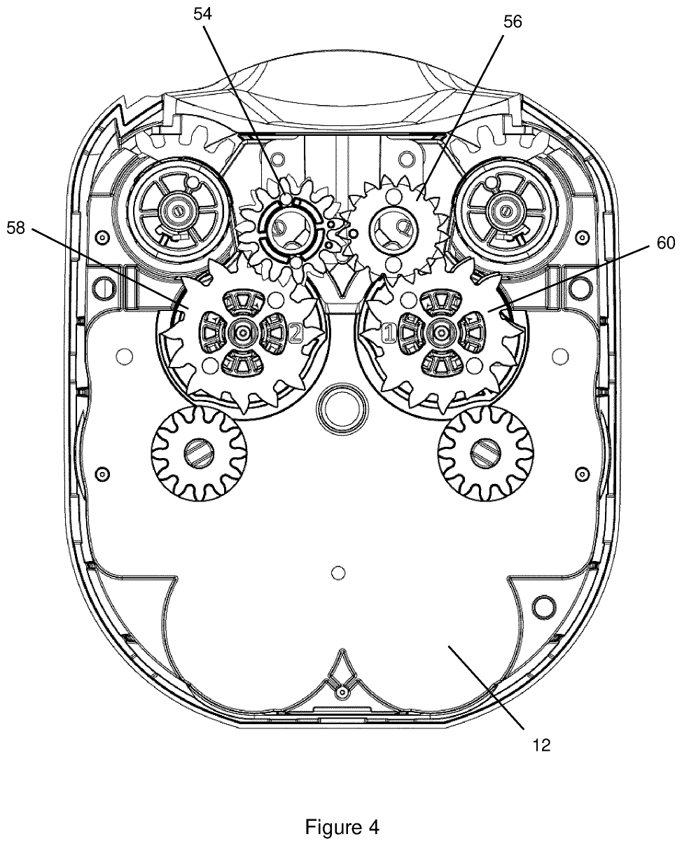

FIG. 4 shows a second part of the mechanism from the device of FIGS. 1A and 1B;

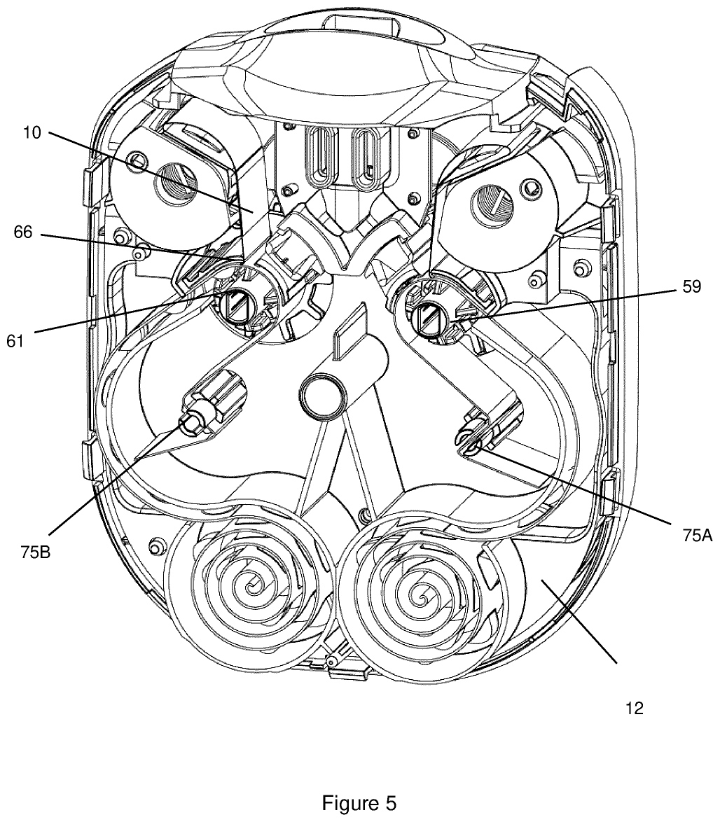

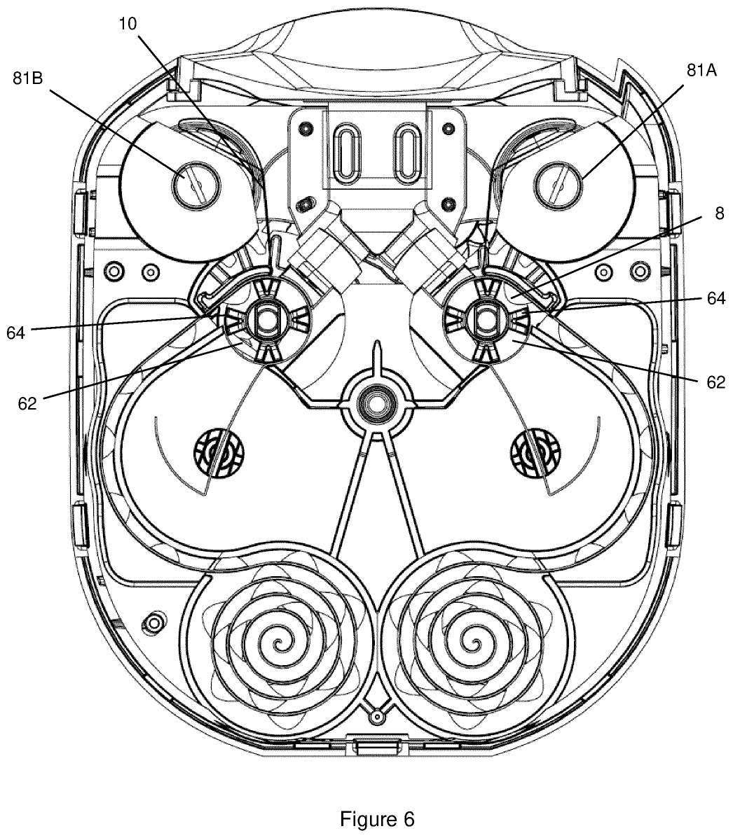

FIGS. 5 and 6 show a medicament blister strip contained within the device of FIGS. 1A and 1B;

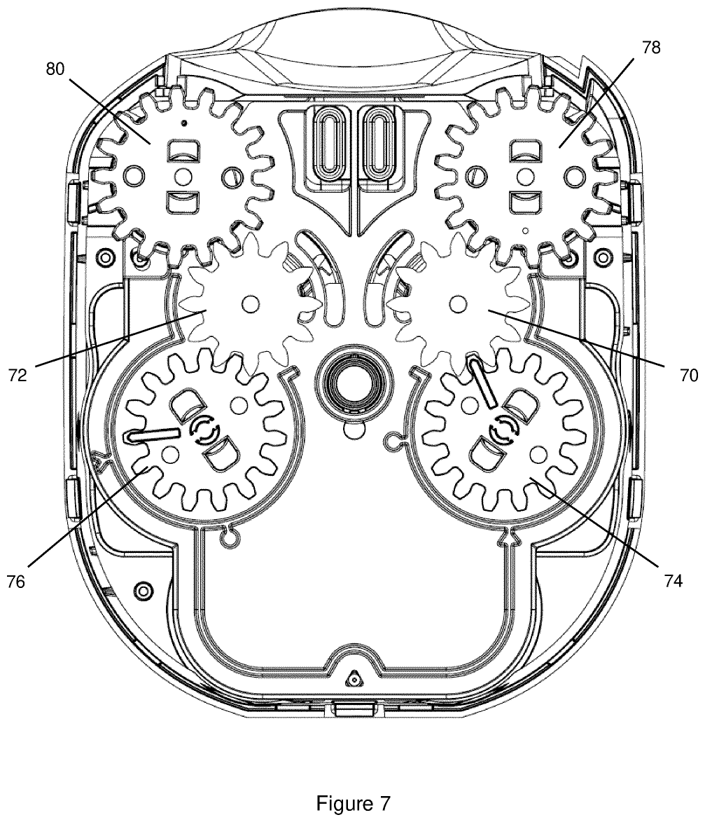

FIG. 7 shows a third part of the mechanism from the device of FIGS. 1A and 1B;

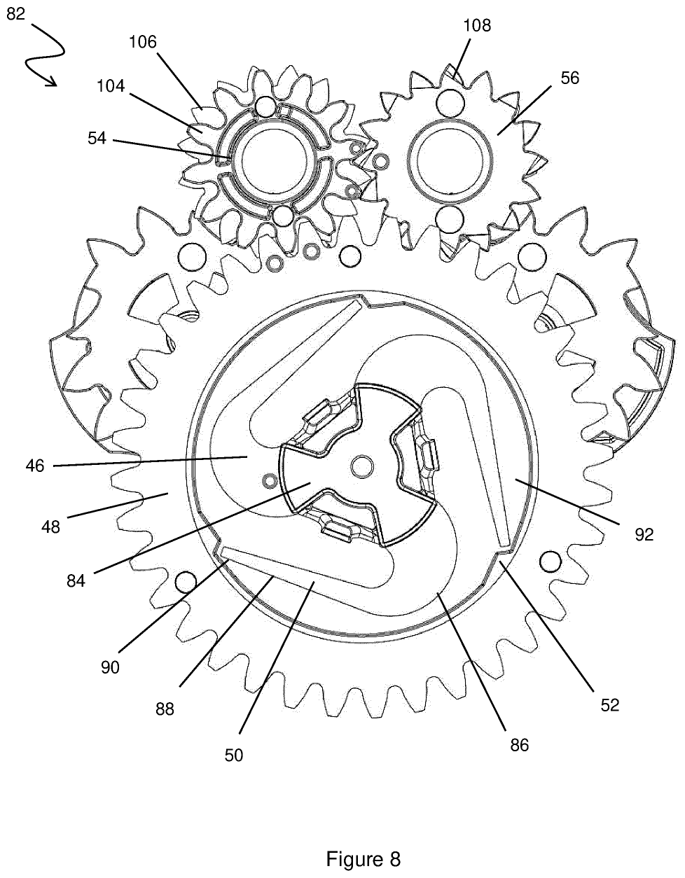

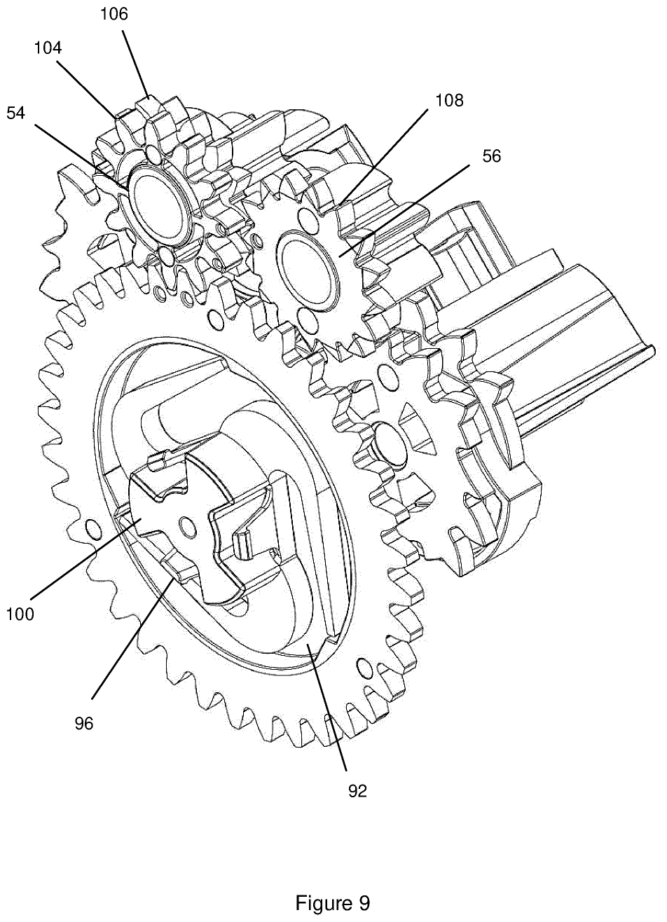

FIGS. 8 and 9 show the arrangement and interaction of the first and second parts of the mechanism from FIGS. 3A, 3B and 4 from a first side;

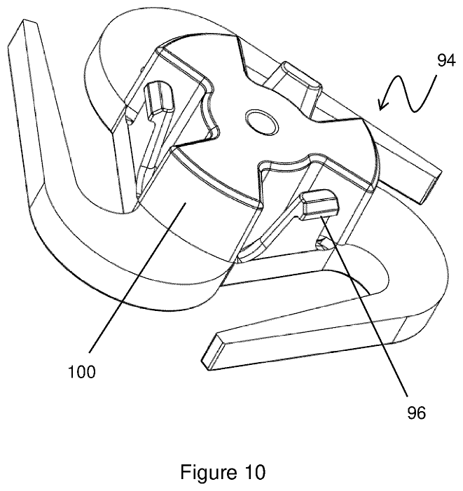

FIG. 10 shows an isolated drive component from FIGS. 8 and 9;

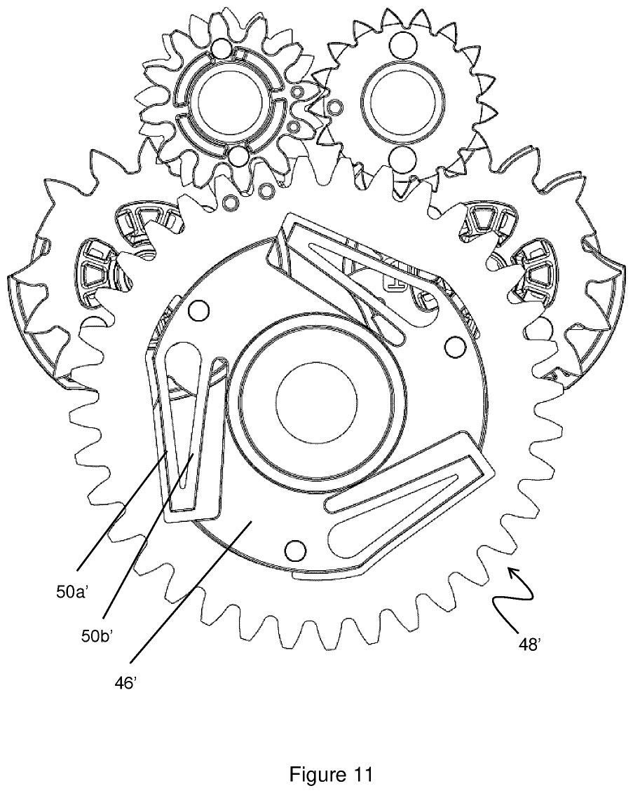

FIG. 11 shows the interaction of alternative first and second parts of a mechanism, as shown in FIGS. 2A and 2B;

FIGS. 12 and 13 show an isolated drive component from FIG. 11;

FIG. 14 shows a part-perspective view of the mouthpiece cover from FIGS. 2A and 2B;

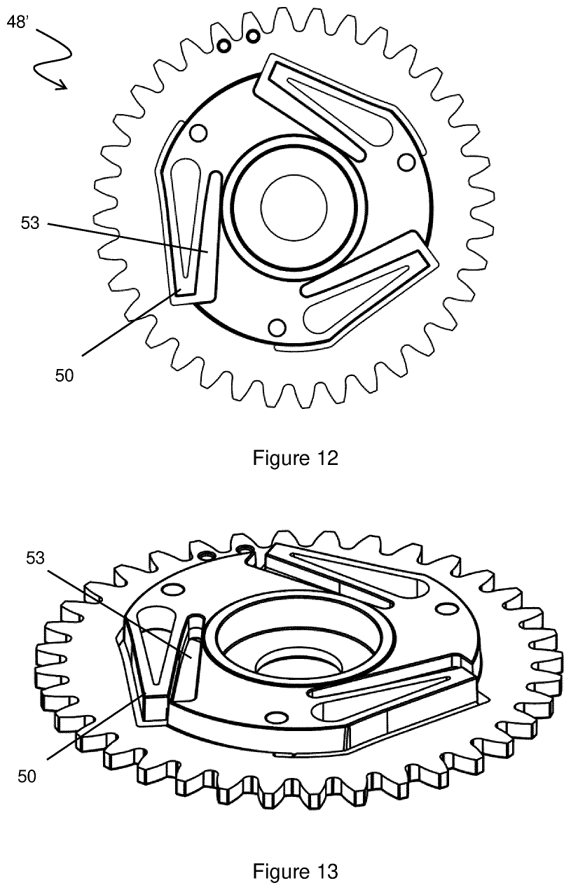

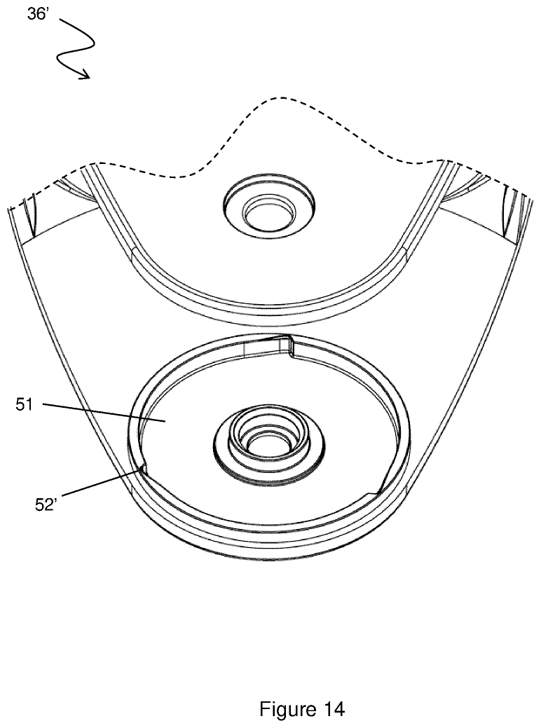

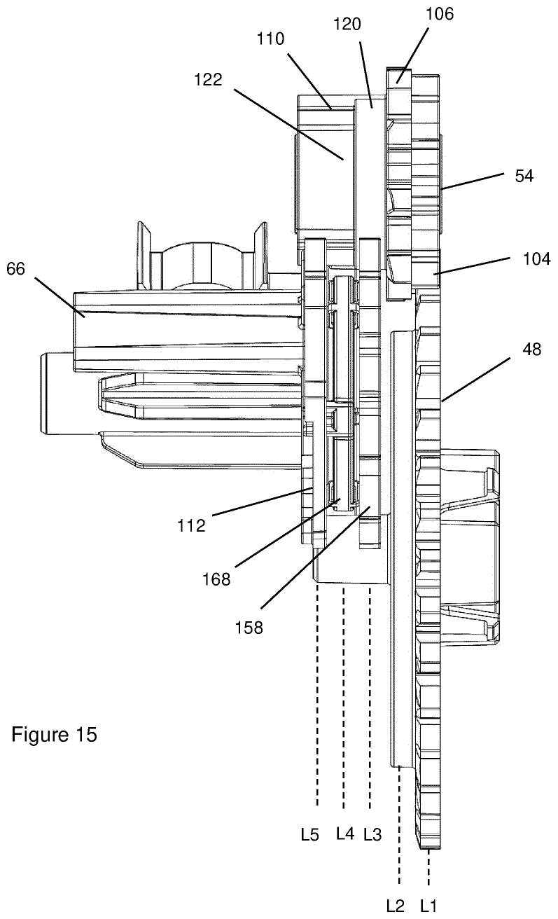

FIG. 15 shows an edge view of the arrangement from FIGS. 8 and 9;

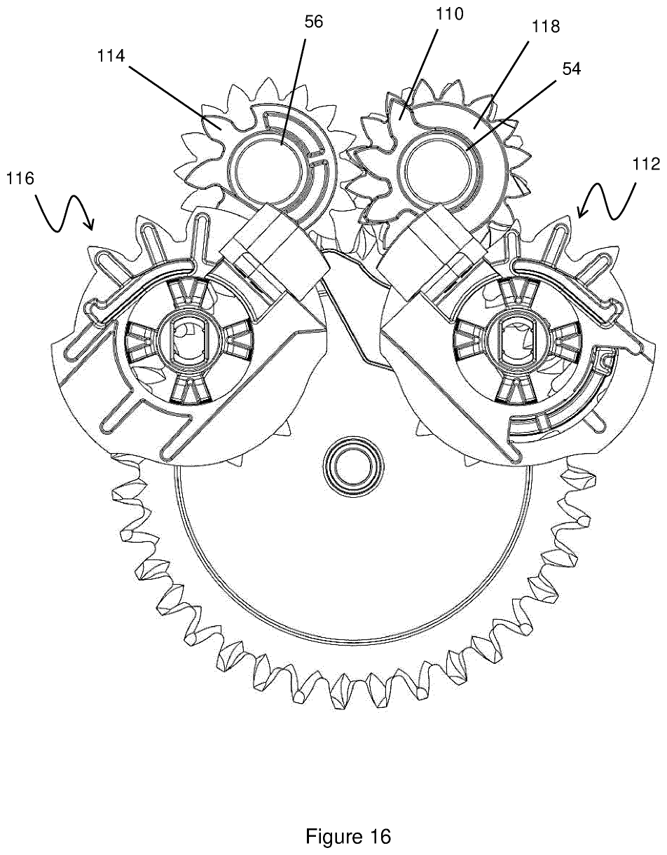

FIGS. 16 to 18 show the arrangement from FIGS. 8 and 9 from a second, opposite, side;

FIGS. 19A and 19B show first examples of index gears for an inhaler device;

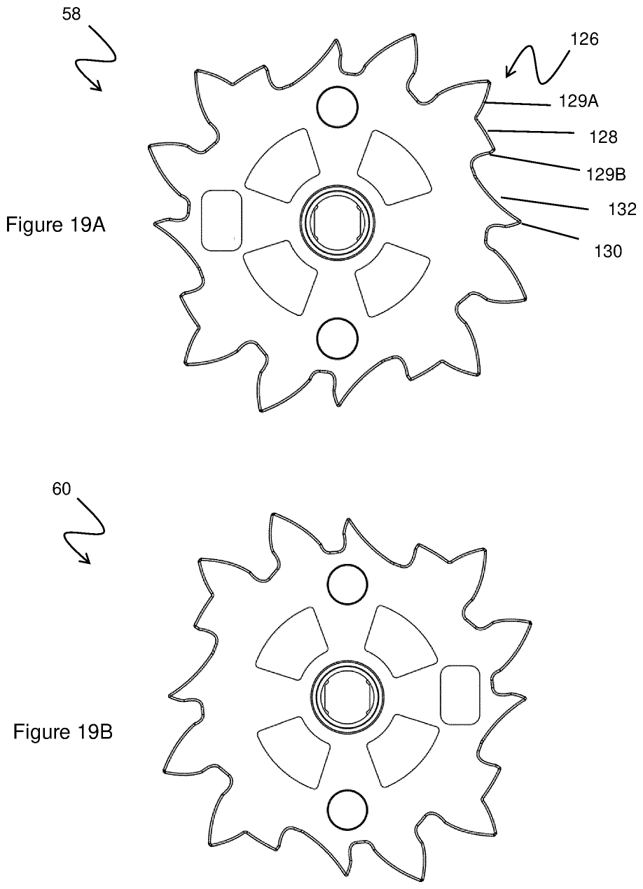

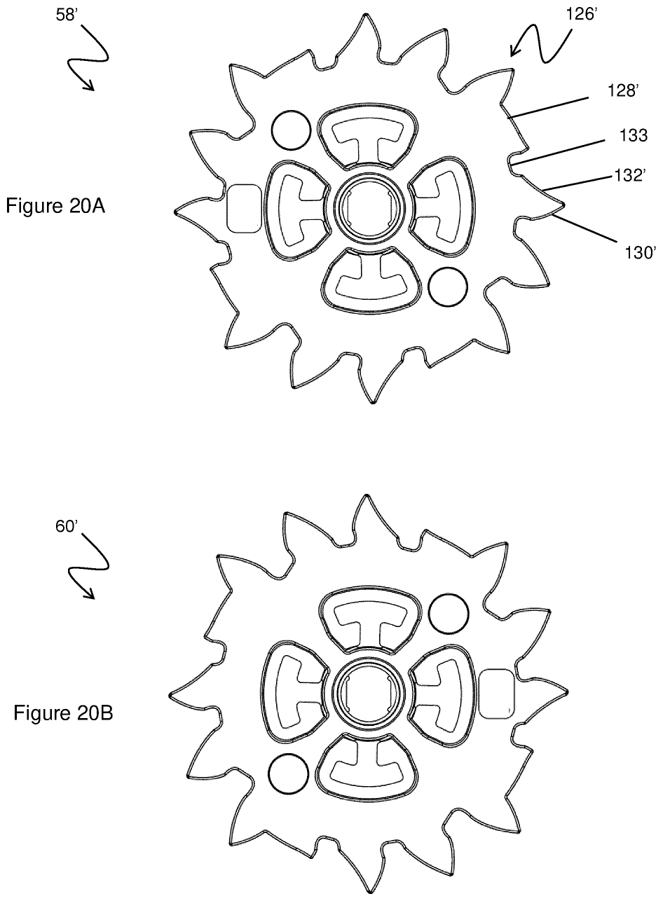

FIGS. 20A and 20B show second examples of index gears;

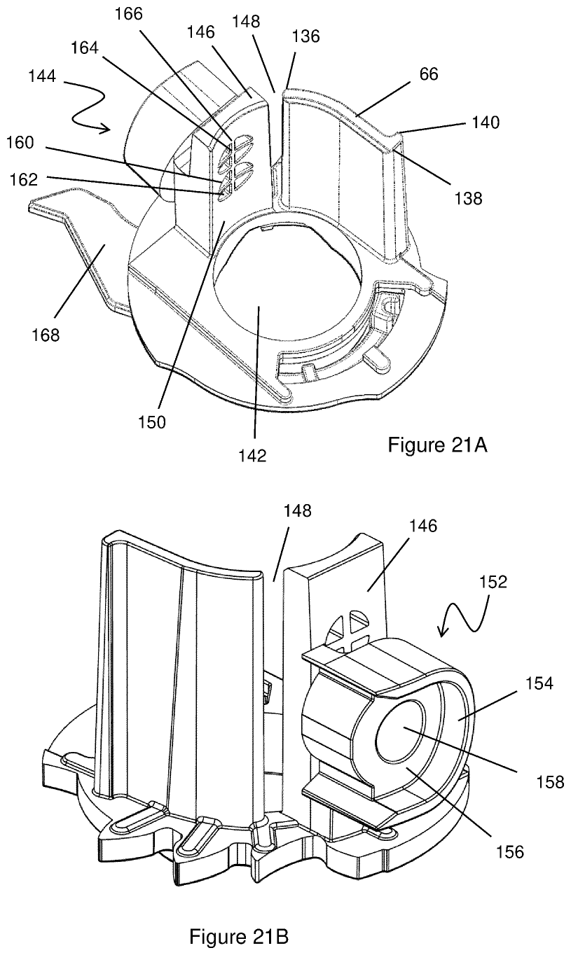

FIG. 21A shows a peel beak component and an interlock component from an inhaler device;

FIG. 21B shows the peel beak component of FIG. 21A in isolation;

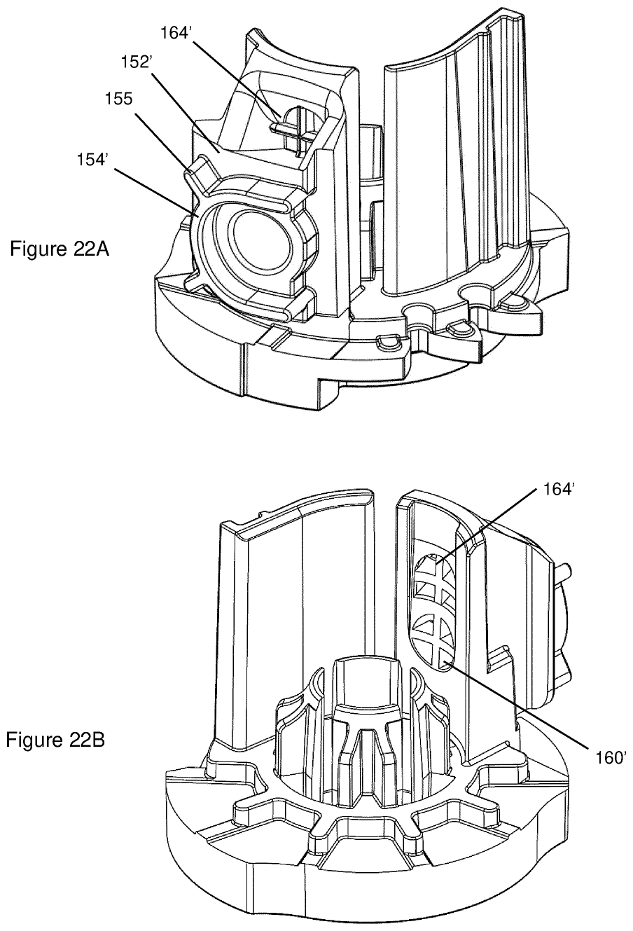

FIGS. 22A and 22B show an alternative peel beak component;

FIG. 23A shows the interlock component of FIG. 21A in isolation;

FIG. 23B shows an opposite angle of the peel beak component and interlock component shown in FIG. 21A;

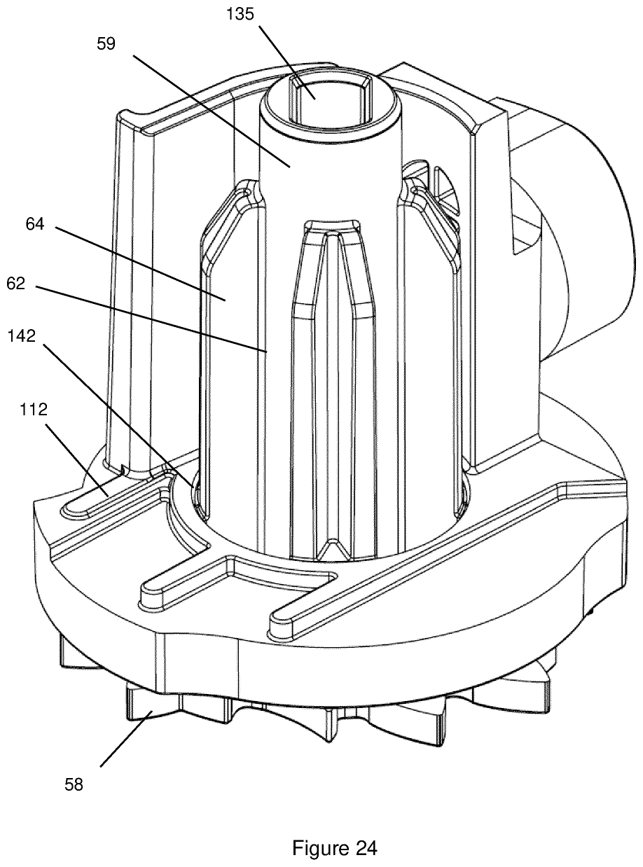

FIGS. 24 and 25 show the assembly of the peel beak component from FIG. 21B with an indexing component;

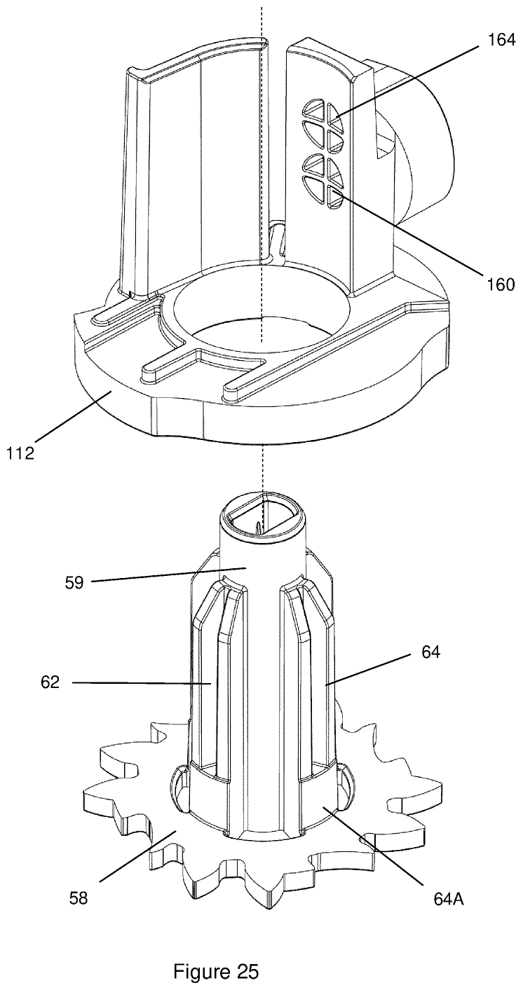

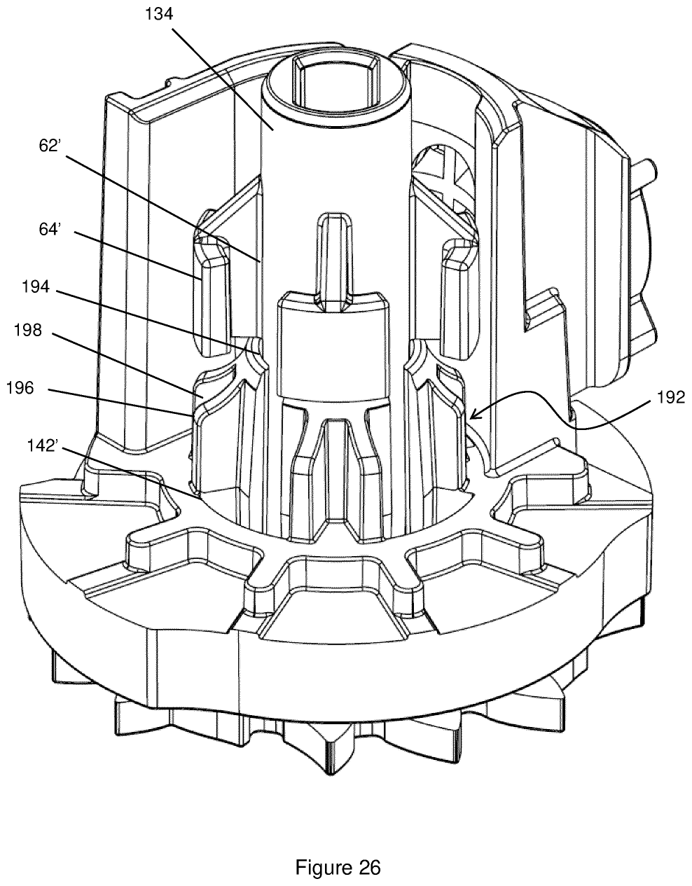

FIG. 26 shows the peel beak component from FIGS. 22A and 22B engaged with an alternative indexing component;

FIG. 27 shows the indexing component from FIG. 26 in isolation;

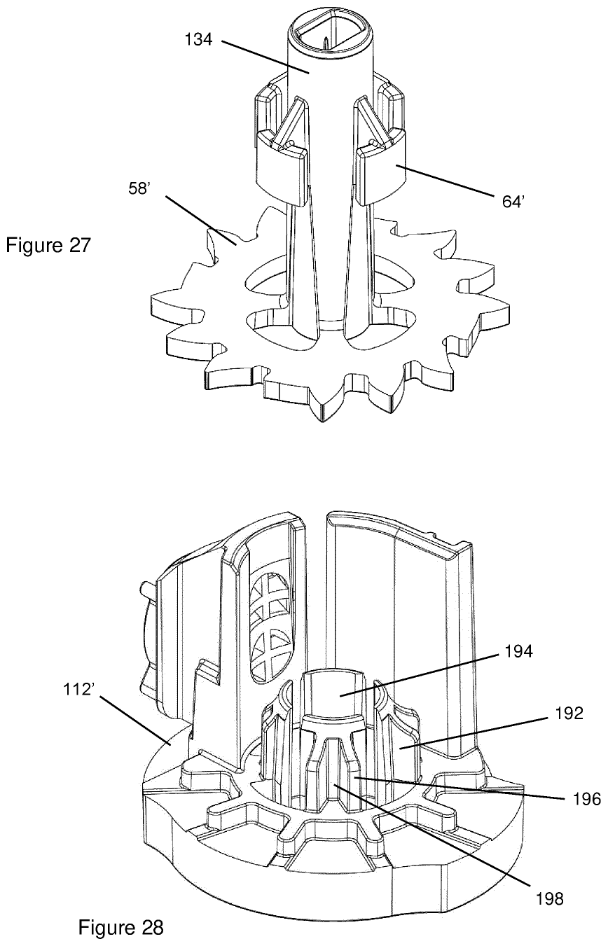

FIG. 28 shows the peel beak component from FIG. 26 in isolation;

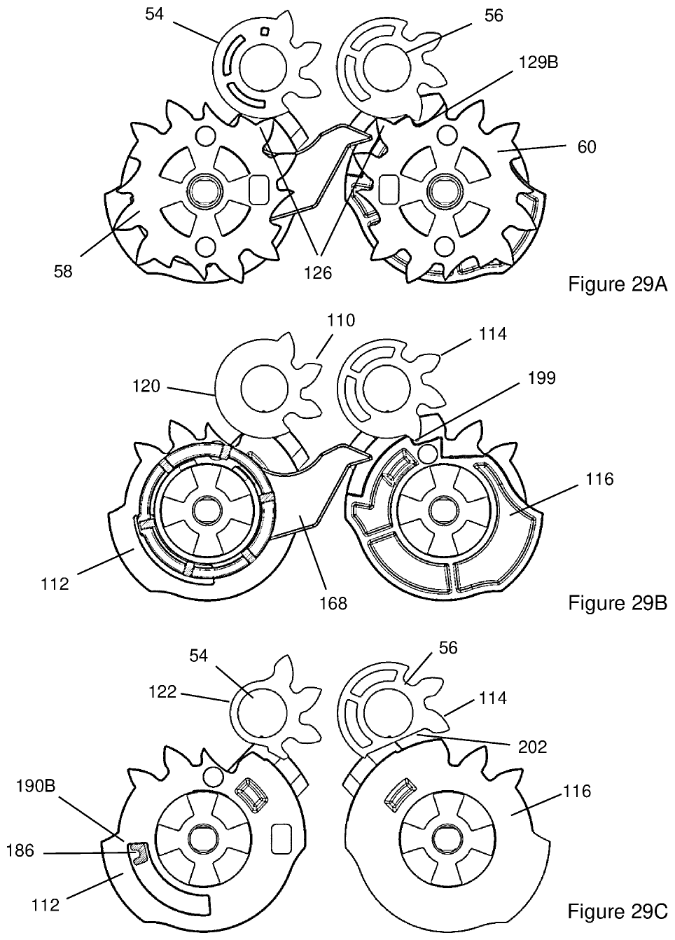

FIGS. 29A to 34C illustrate the operation of various levels of the gear arrangement from FIG. 15 during use;

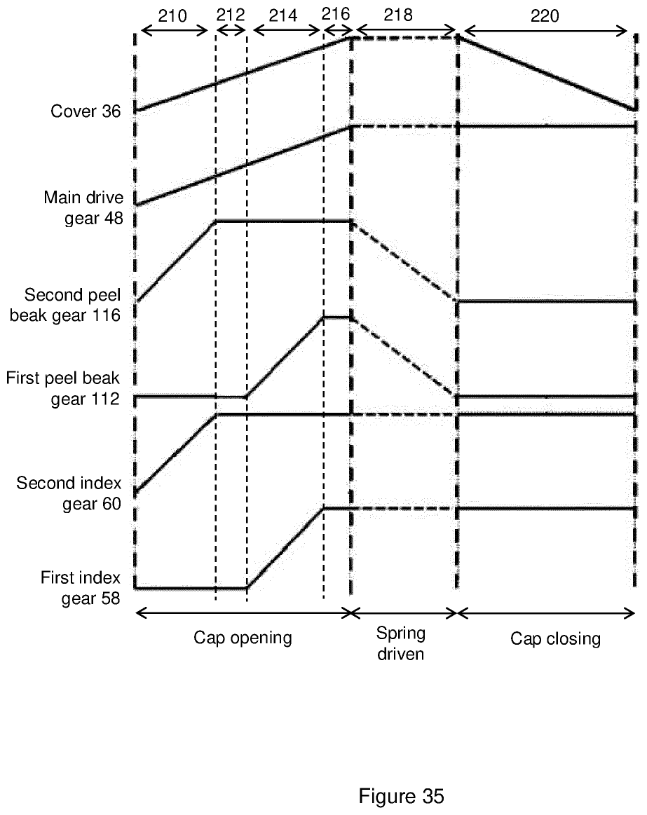

FIG. 35 shows a graphical representation of the movement of various components during the movement shown in FIGS. 29A to 34C;

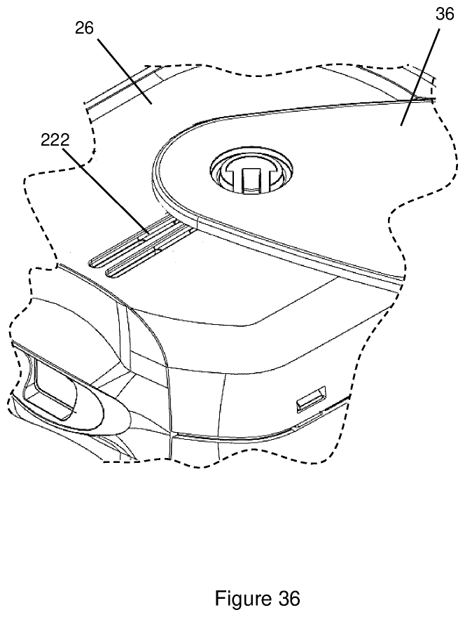

FIG. 36 shows a partial view of the outside of an inhaler device with a mouthpiece cover open;

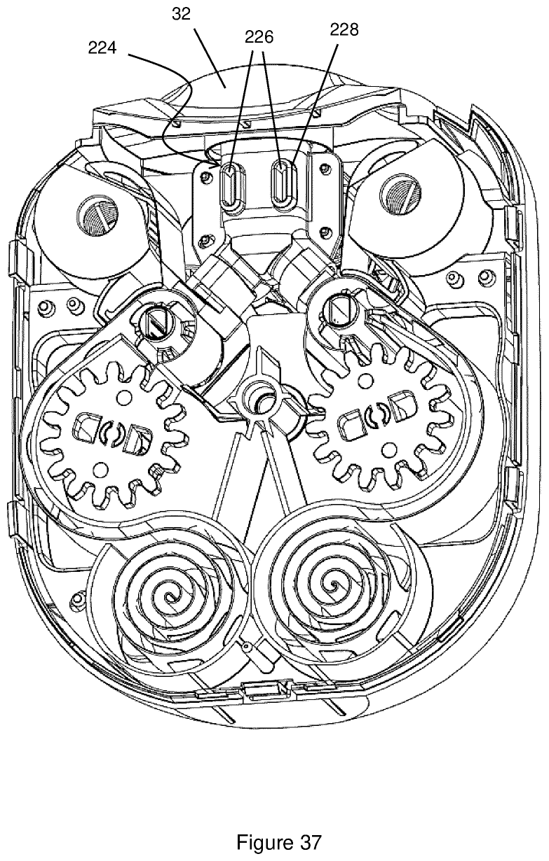

FIG. 37 shows a view, similar to FIG. 5, of the interior of an inhaler device with a blister strip shown;

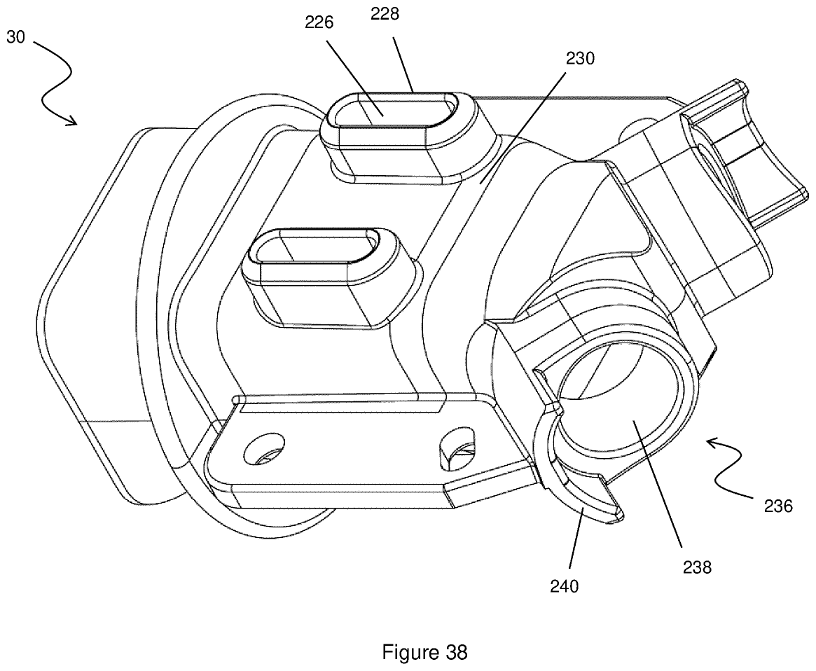

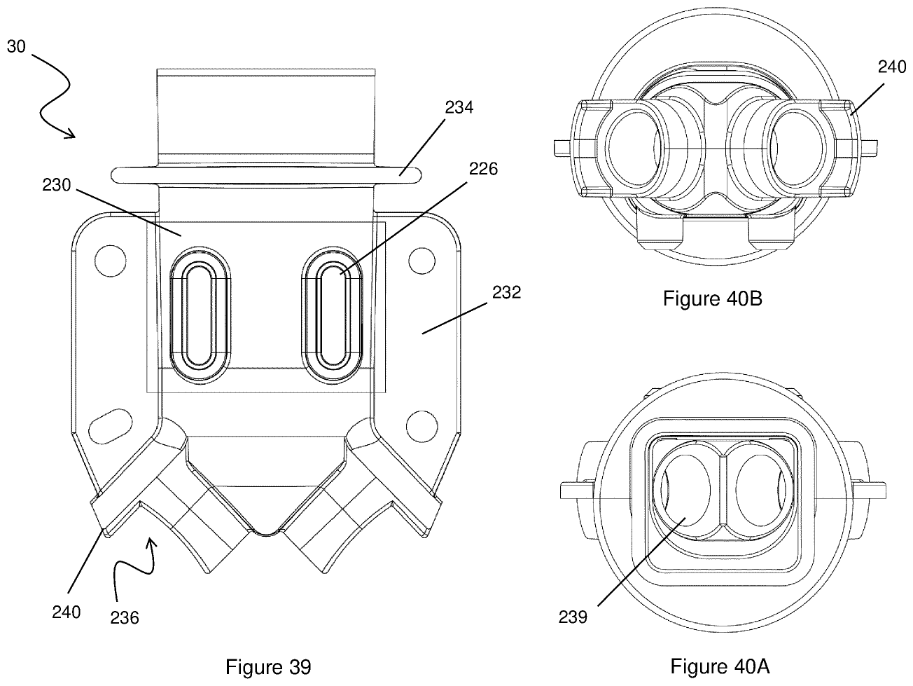

FIGS. 38 to 40B show an airway manifold component for an inhaler device;

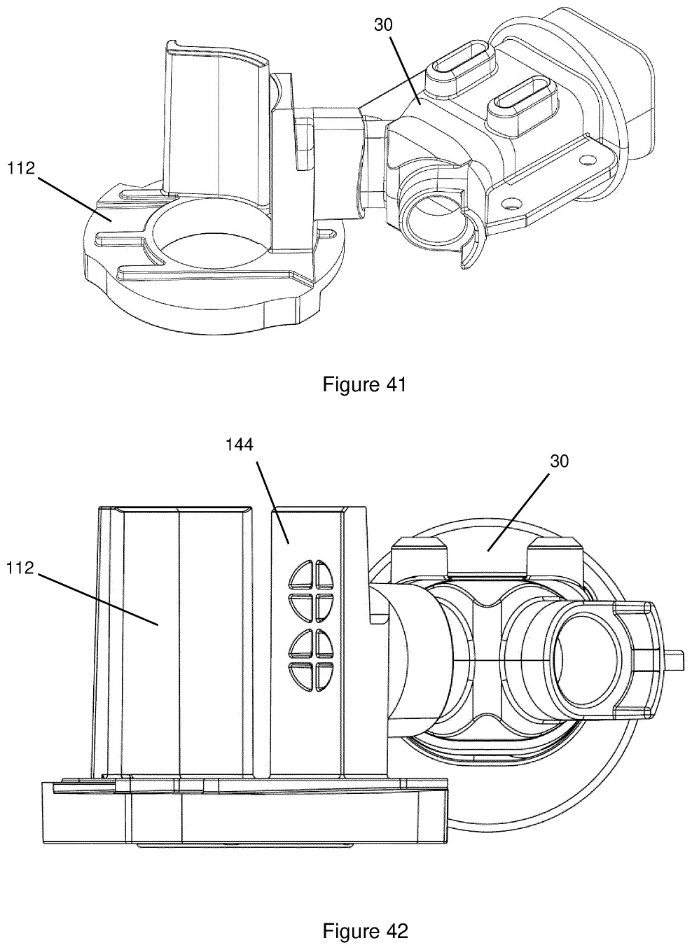

FIGS. 41 and 42 show the interaction of a peel beak component with the airway manifold component from FIG. 38;

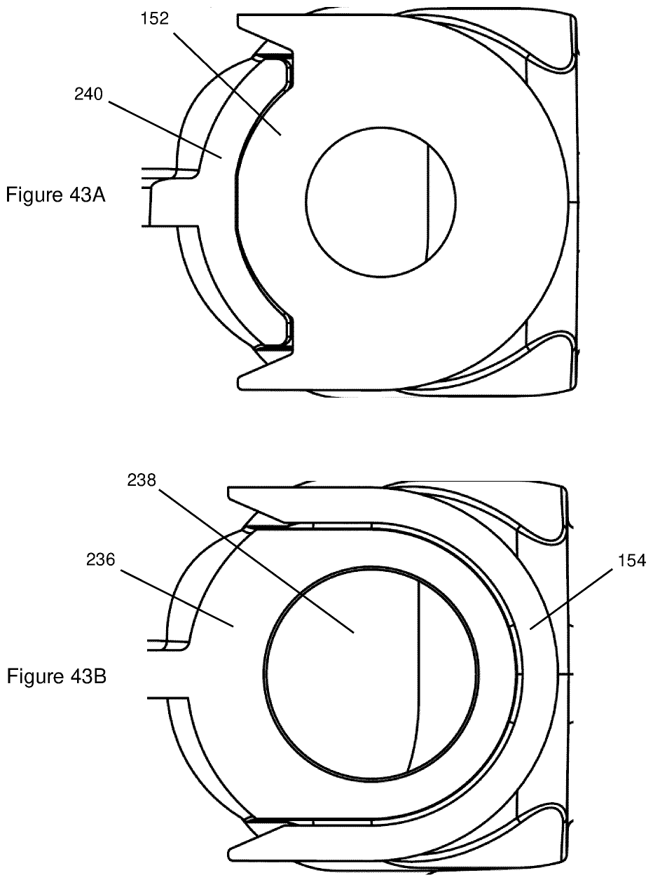

FIGS. 43A and 43B show the sealing of part of a peel beak component with the airway manifold component from FIG. 38;

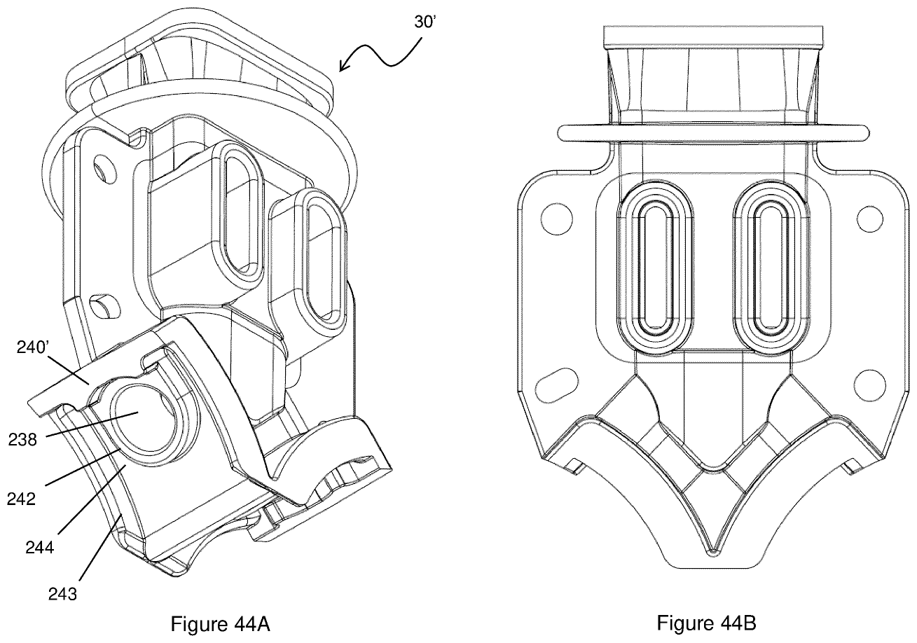

FIGS. 44A and 44B show an alternative airway manifold component;

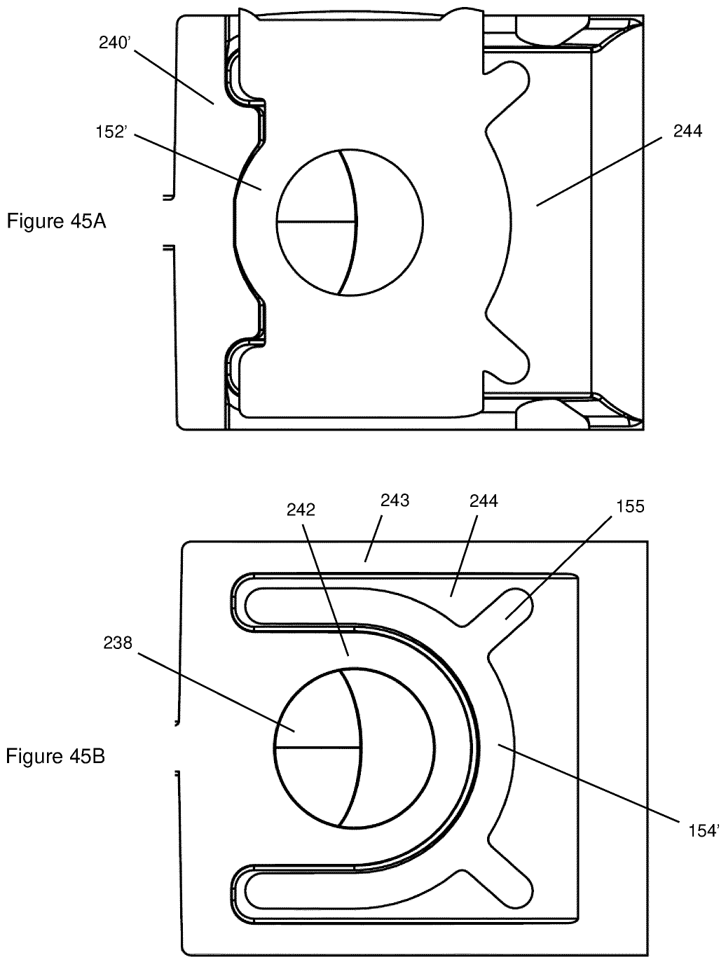

FIGS. 45A and 45B show the sealing of part of a peel beak component with the airway manifold component from FIGS. 44A and 44B;

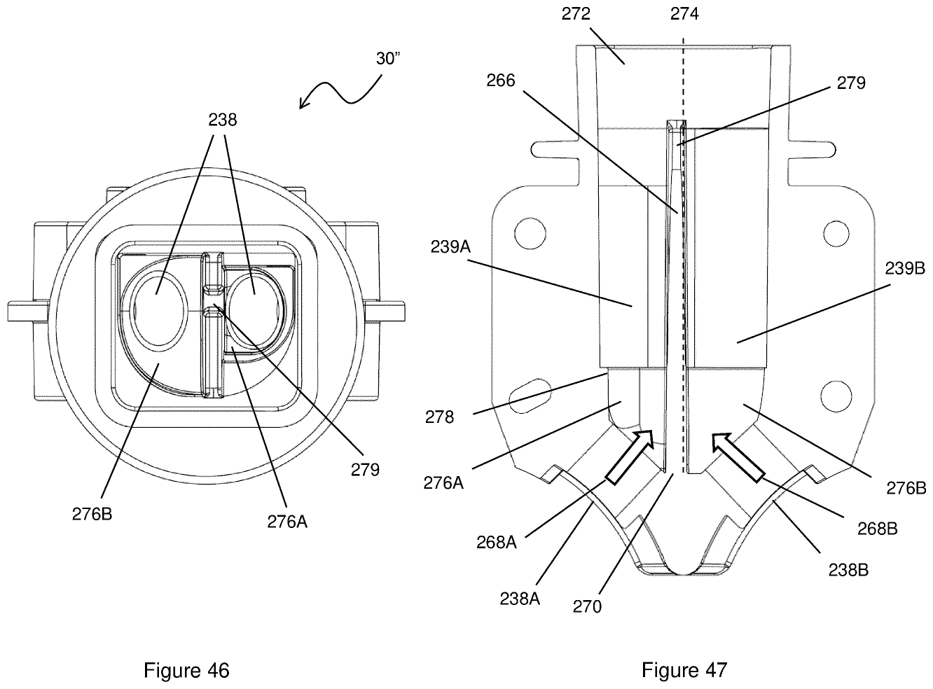

FIGS. 46 and 47 show a further alternative airway manifold component;

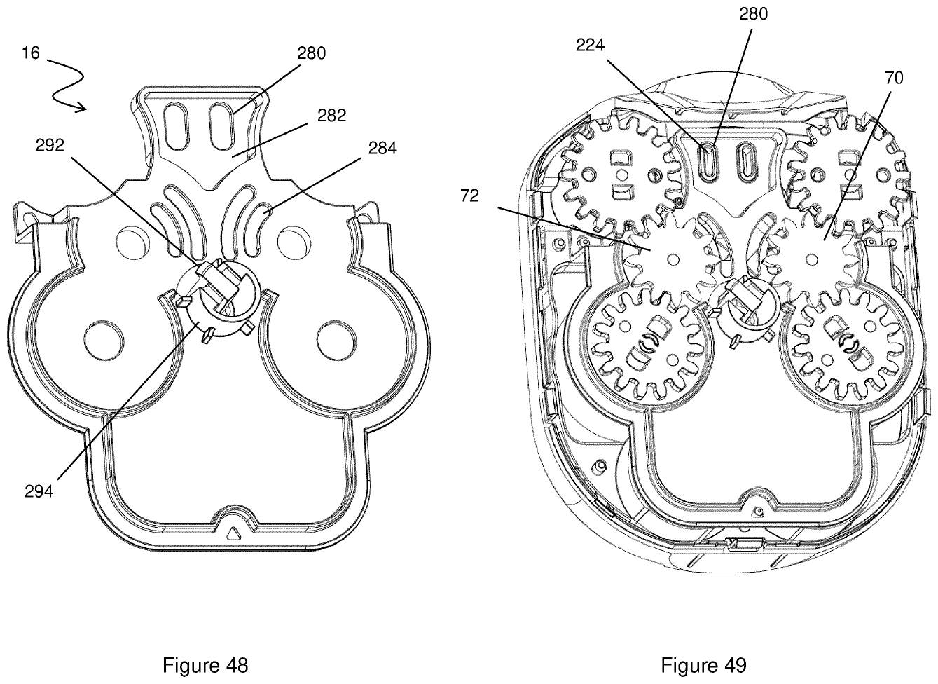

FIG. 48 shows an intermediate chassis component from the inhaler device of FIGS. 1A and 1B;

FIG. 49 shows the intermediate chassis component of FIG. 48 within an inhaler device;

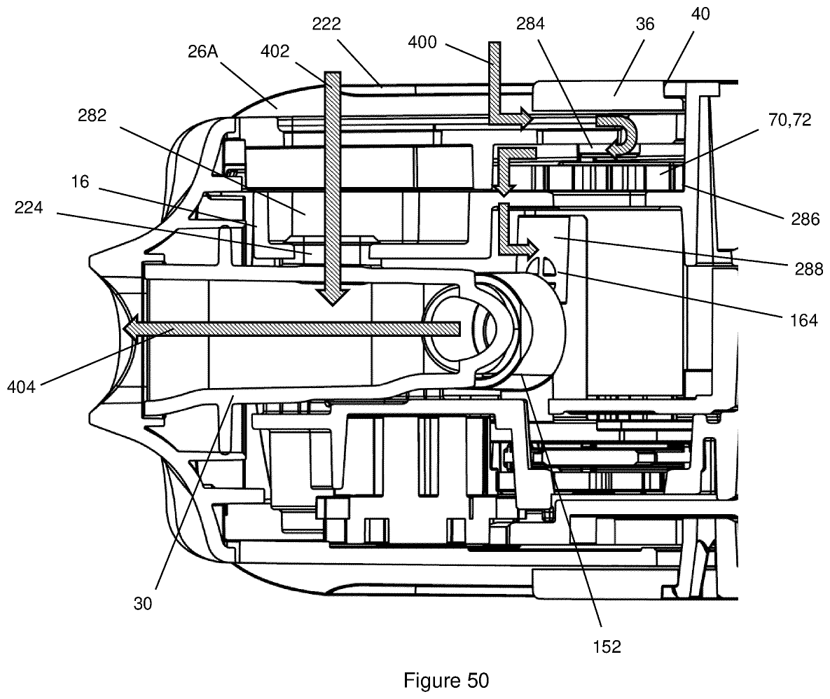

FIGS. 50 to 52 illustrate airflow through an inhaler device;

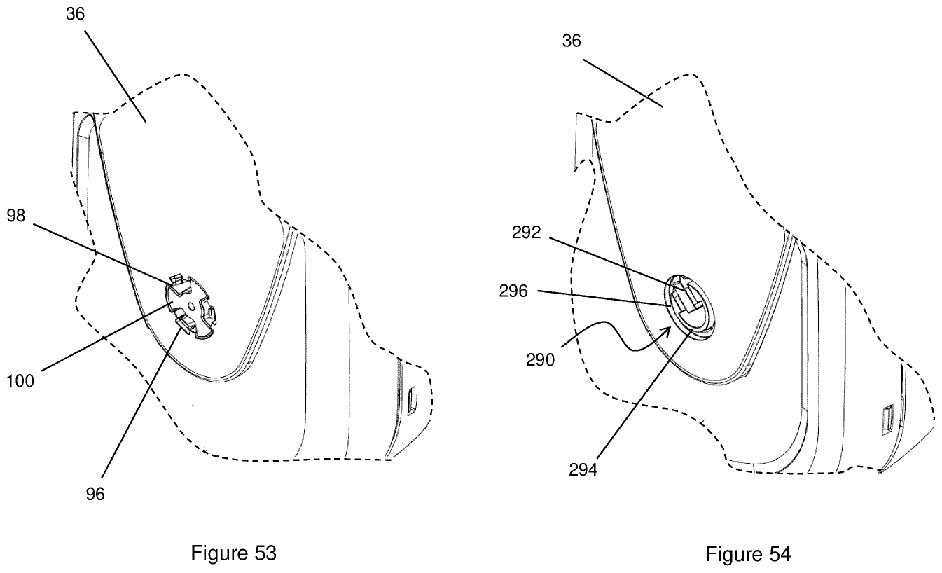

FIGS. 53 and 54 show partial views of the outside of an assembled inhaler device;

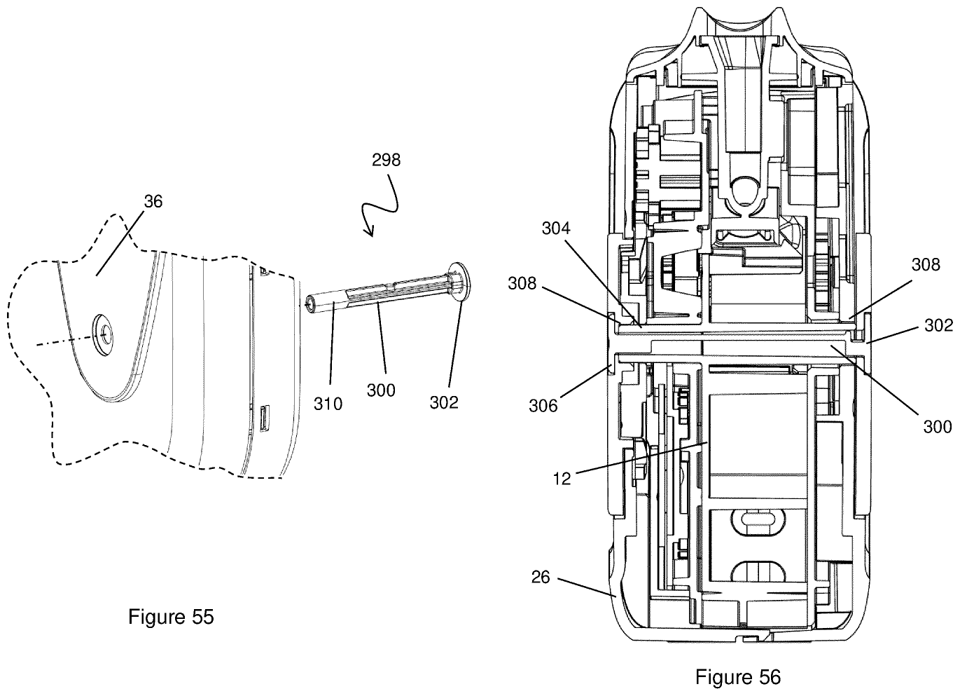

FIG. 55 shows a partial view of the outside of an alternative inhaler device;

FIG. 56 shows a cross section take through the inhaler device of FIG. 55;

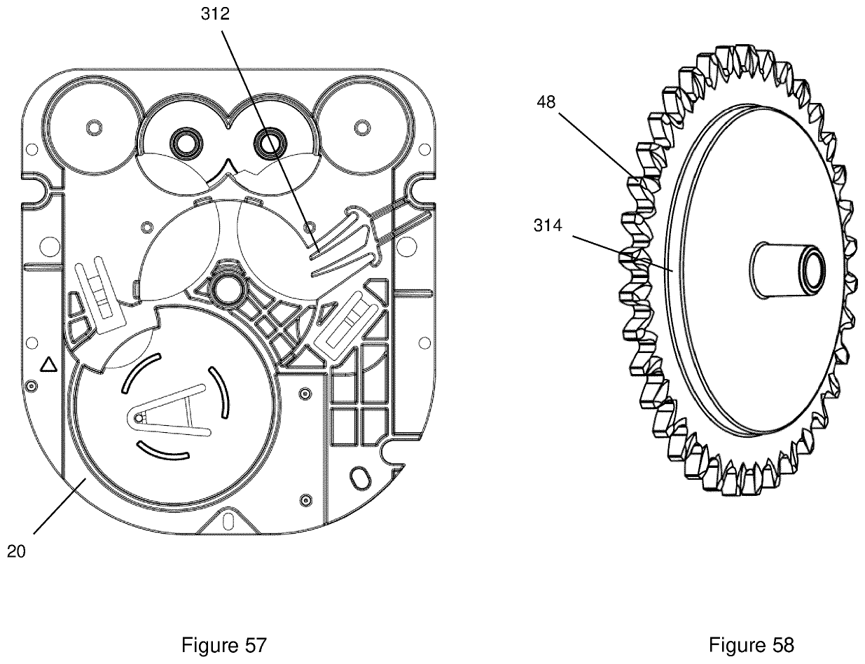

FIG. 57 shows another intermediate chassis component from the inhaler device of FIGS. 1A and 1B;

FIG. 58 shows a rear face of a drive component from the inhaler device of FIGS. 1A and 1B;

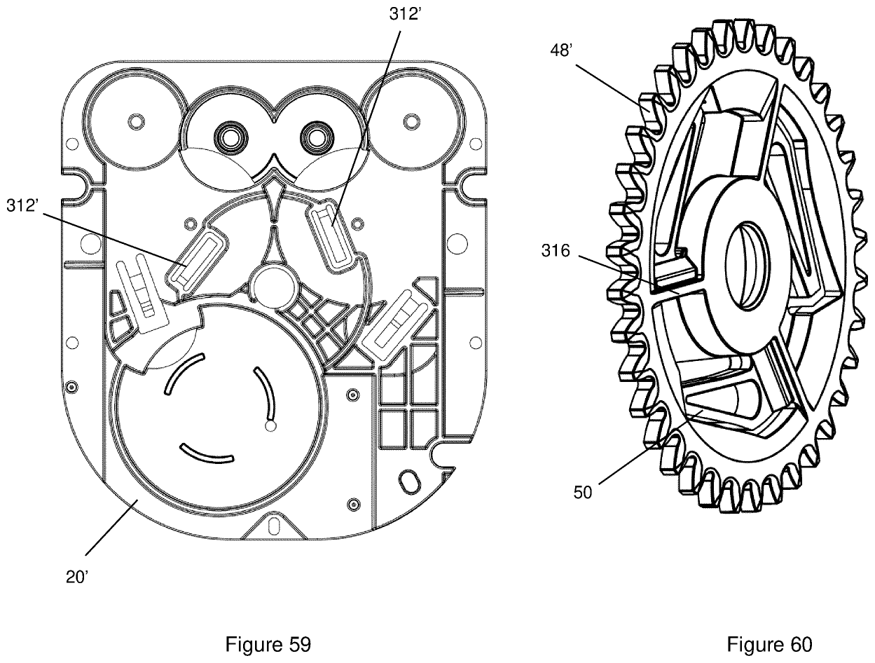

FIG. 59 shows an alternative intermediate chassis component from the inhaler device of FIGS. 2A and 2B;

FIG. 60 shows a rear face of the drive component from FIGS. 12 and 13;

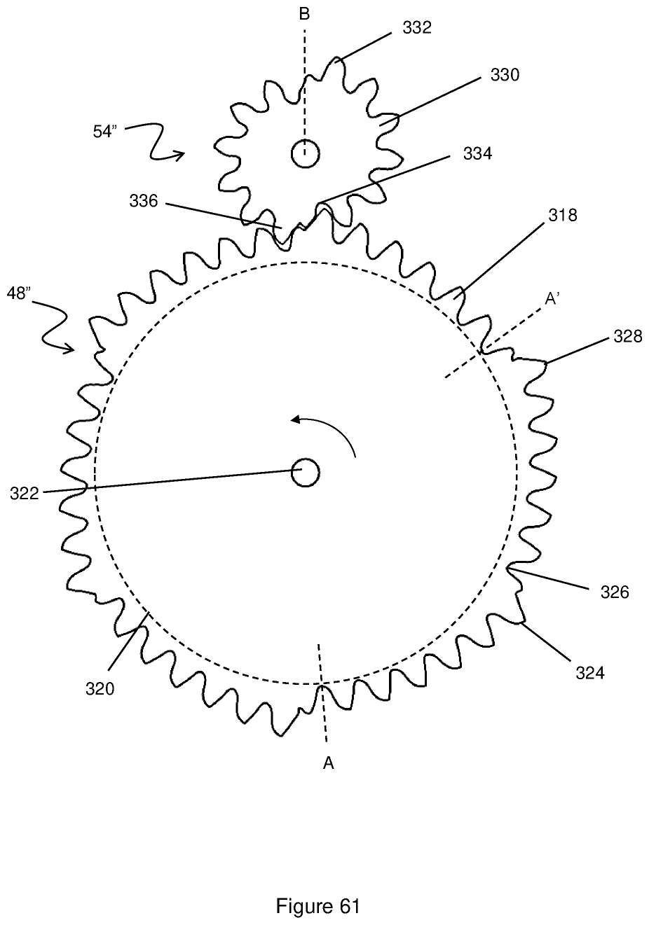

FIG. 61 shows a pair of gears for use in an inhaler device;

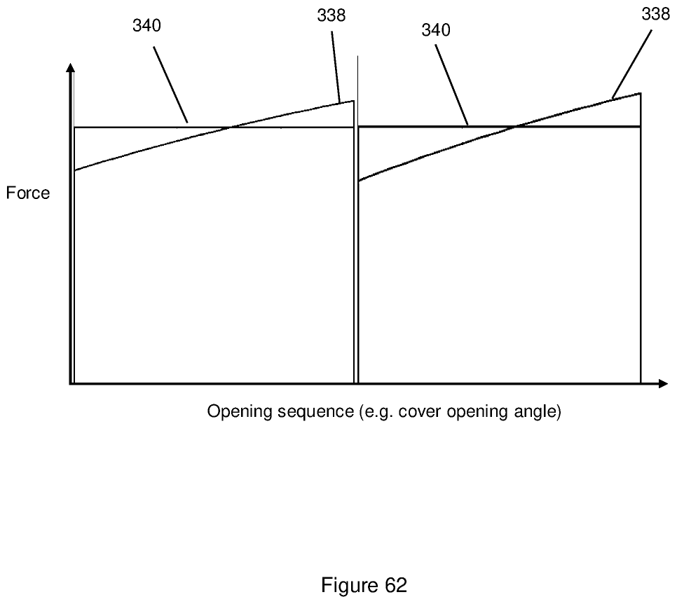

FIG. 62 shows a plot of input force during operation of two different inhaler devices;

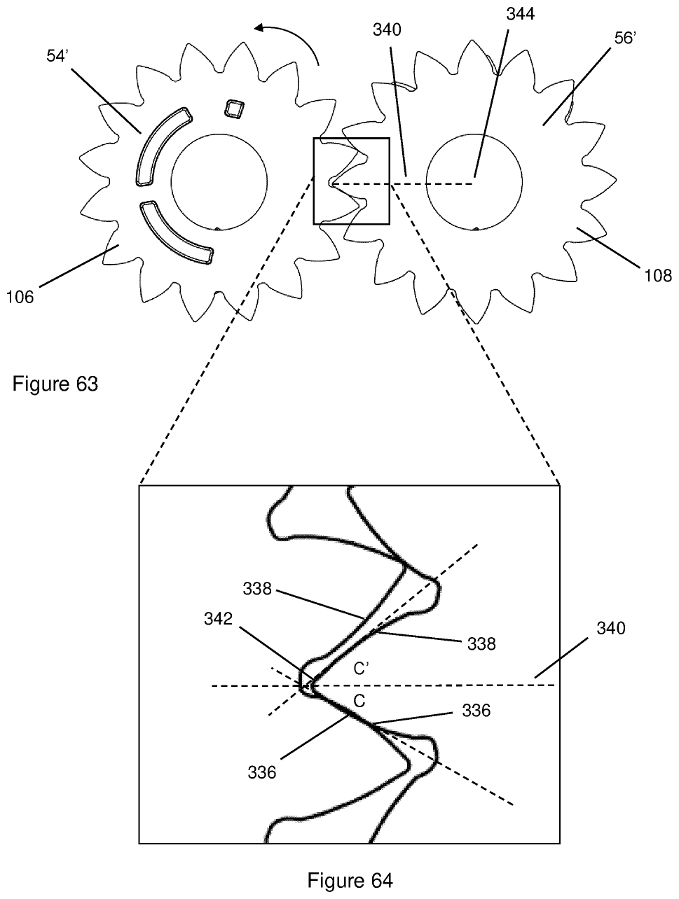

FIG. 63 shows another pair of gears for use in an inhaler device; and

FIG. 64 shows a detail view of the interaction of gear teeth from FIG. 63.

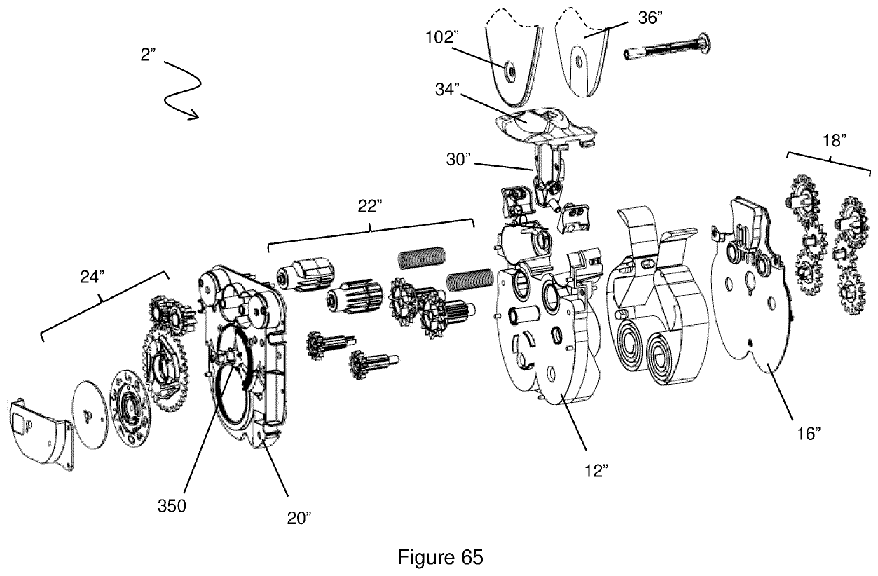

FIGS. 65 and 66 show exploded views of a third example inhaler device;

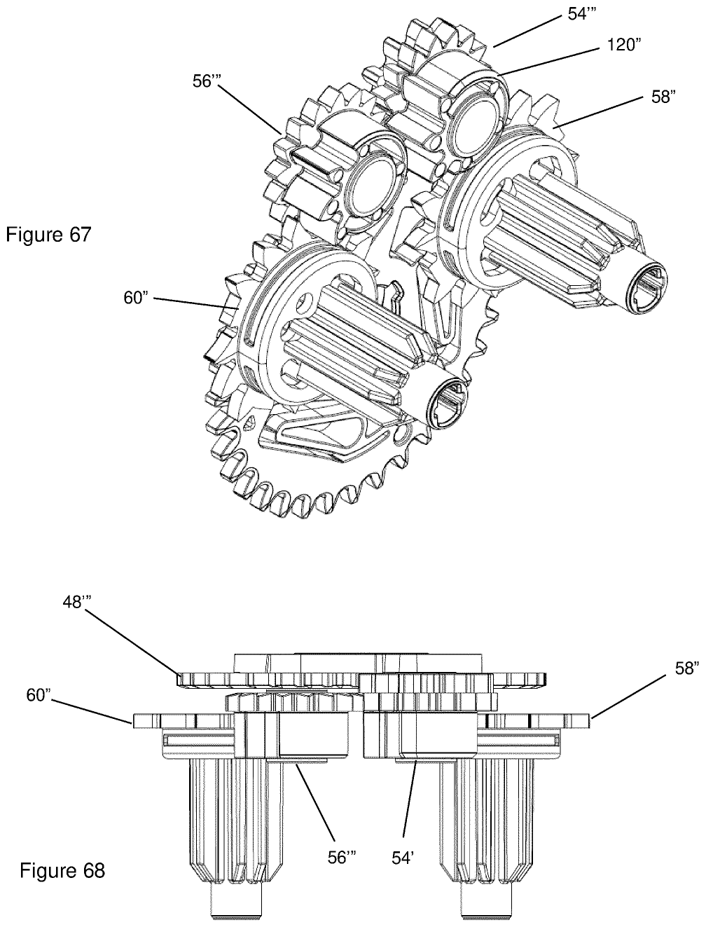

FIG. 67 shows a perspective view of the third gear mechanism of the third example;

FIG. 68 shows a top-down view of a third gear mechanism of the third example;

FIG. 69 shows a perspective view of a first drive gear of the third example;

FIG. 70 shows a top-down view of a main drive gear mechanism of the third example;

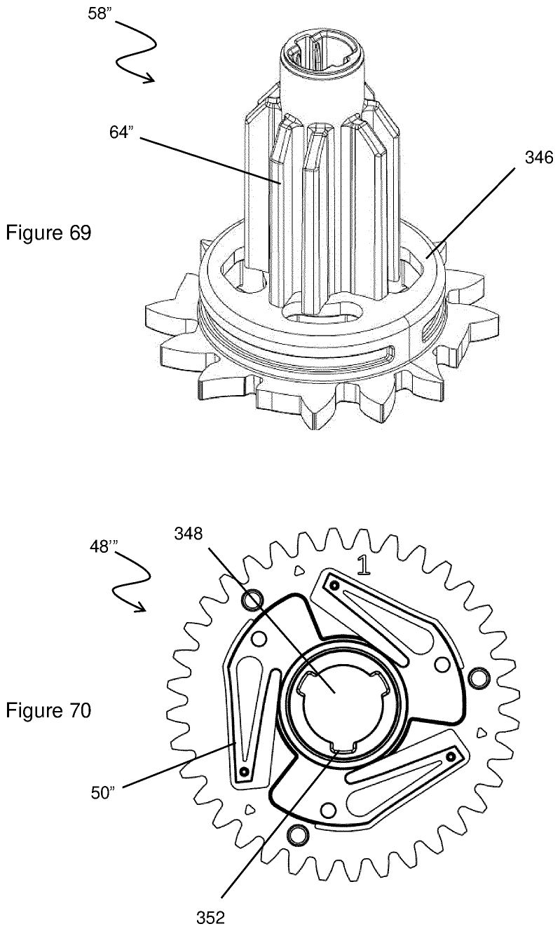

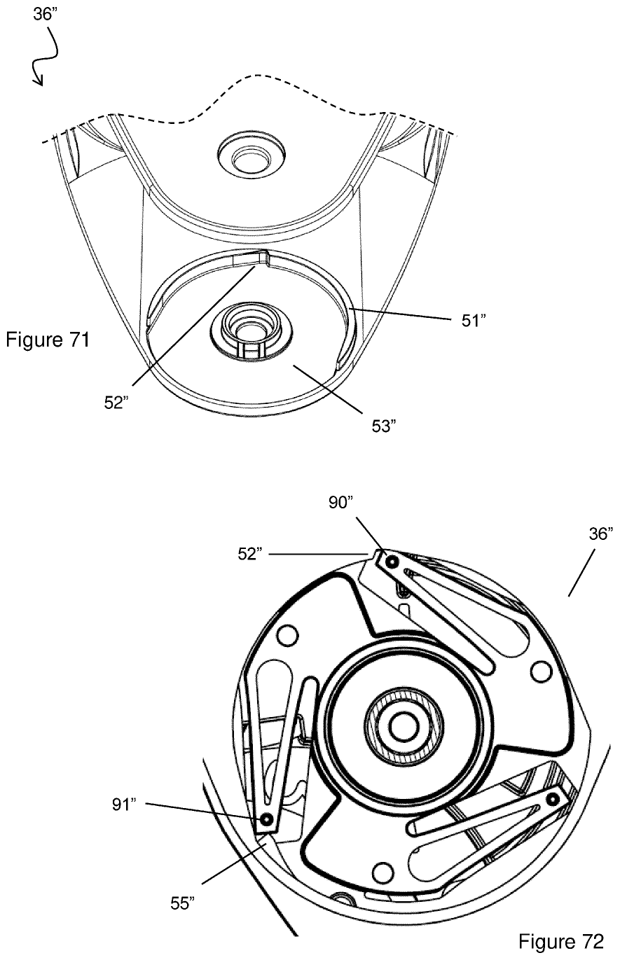

FIG. 71 shows a perspective view of a cap of the third example;

FIG. 72 shows a cross sectional view of the interface of the cap of FIG. 71 with a housing component;

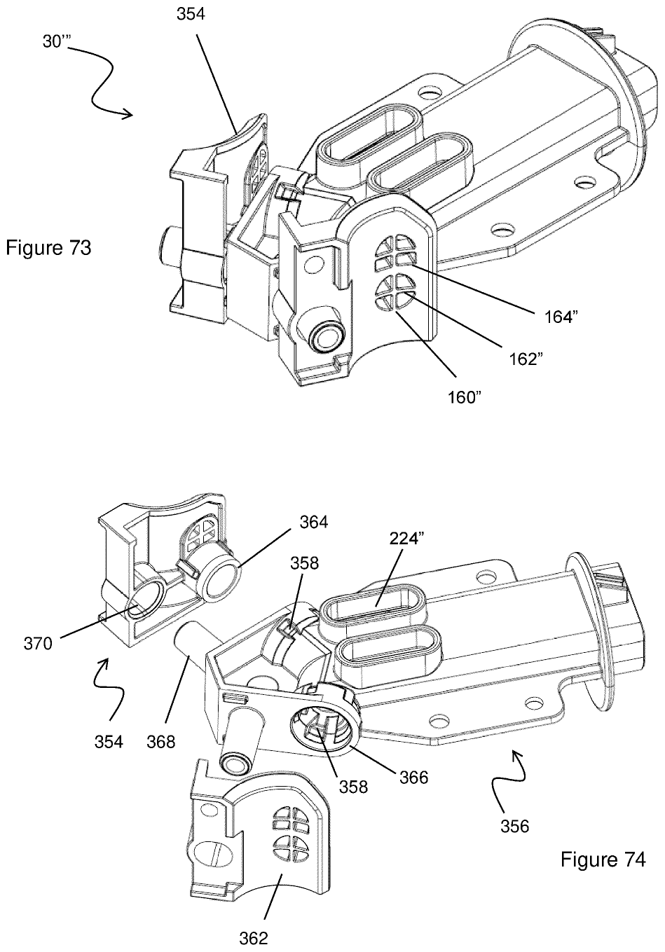

FIGS. 73-75 show perspective views of a manifold assembly of the third example; and

FIGS. 76 and 77 show perspective views of a take-up drum of the third example.

### OVERVIEW

FIGS. 1A-B and 3-6 show the interior of an inhaler device 2 according to a first embodiment of the invention. The inhaler device 2 is configured to use elongate blister strips 4 of medicament comprising a base sheet/foil 6, defining individual blister pockets 8 of medicament, and a lidding sheet 10 in the form of a lidding foil which is peeled from the base foil 6 to open the pockets and expose the medicament for inhalation.

A strip chassis 12 defines interior chambers and pathways of the device for receiving and guiding the blister strips 4. The strip chassis 12 comprises a plurality of upstanding walls 14 configured to define the various pathways and compartments. The strip chassis 12 may comprise a single moulded piece. A chassis cover 16 is configured to overlie the strip chassis 12, thus at least partially enclosing the compartments/pathways defined by the strip chassis 12. The chassis cover 16 supports a first gear mechanism 18.

A gear chassis 20 supports a second gear mechanism 22 and a third gear mechanism 24. The gear chassis 20 may be fixed or otherwise mounted or the strip chassis 12.

A housing 26 encloses the chassis 12,20, the cover 16 and the gear mechanisms 18,22,24. The housing 26 is formed from two shell portions 26A,26B united to form a single housing. The housing portions 26A,B may be secured to each other by a snap or interference fit, or by any other suitable securing method. The housing portions 26A,26B comprise a latch mechanism 28 to prevent unauthorised deconstruction or tampering of the device. The exact form of housing 26 is not pertinent to the invention at hand. In some embodiments, the housing may only partially enclose the gear mechanisms 18,22,24 etc. However, it can be appreciated that a complete housing prevents interference with the operation of the device.

The device 2 comprises an airway manifold, at least part of which 30 is fixed to the gear chassis 20. The fixed part of the airway manifold 30 is fluidly connected to a mouthpiece 32. The mouthpiece 32 comprises a raised lip structure 34 configured to be received by the lips of the user in use (see FIG. 1B). The mouthpiece may be sandwiched between the two housing portions 26A,B.

A mouthpiece cover 36 is configured to cover the mouthpiece 32 in use (e.g. in a storage configuration). The cover 36 is rotatable between a first position where the mouthpiece 32 is covered and second position where the mouthpiece 32 is uncovered. The cover 36 may therefore protect the mouthpiece from the ingress or dirt and/or dust etc. The cover 36 is rotatable about an axis 38. The axis passes through the approximate centre of the device 2. The cover 36 is configured to be mounted into/onto one or both apertures 40 provided in the respective housing portions 26A,B. For example, the cover 36 may comprise a latch, protrusion, lip or rim configured to snap fit into the aperture 40. It can be appreciated that such an arrangement is merely exemplary, and the cover may be mounted to the housing 26 in any suitable fashion. A cover panel (not shown) may overlie the side 44 of the cover to prevent the user tampering with the connection between the cover 36 and the housing 26.

A second embodiment of the device 2′ is shown in FIGS. 2A and 2B. The second embodiment is substantially the same as the first embodiment in most aspects of form and operation and like features will not be repeated for the sake of brevity. Similar components are identified with the same reference numeral, sometimes distinguished with a ‘prime’.

The cover 36 is configured to actuate a dispensing mechanism for dispensing a dose of medicament from the blister strip 4. The cover 36 is fixed to a drive pawl 46. As best seen in FIGS. 3A and 3B, the drive pawl 46 is received within a main drive gear 48. The drive pawl 46 comprises a plurality of arms 50 configured to engage engagement formations 52 on the main drive gear 48, such that relative rotation in a first direction is permitted and relative rotation in a second, opposing direction is prevented. Thus, rotation of the cover 36 in the first direction (anticlockwise in FIG. 3) does not drive the main drive gear 48 and rotation of the cover 36 in the second direction (clockwise in FIG. 3) drives the main drive gear 48 in the second direction. Rotation of the main drive gear 48 in thus uni-directional (i.e. the pawl 46 provides a ratchet mechanism). This ensures the dispensing mechanism progresses with each rotation of the cover 36.

A first drive gear 54 meshes with the main drive gear 48. A second drive gear 56 meshes with the first drive gear 54 but not with the main drive gear 48. Rotation of the main drive gear 48 thus drives both the first drive gear 54 and the second drive gear 56, but in opposing rotational directions. As best seen in FIG. 4 the first drive gear 54 and the second drive 56 also mesh/interact/interlock, respectively, with a first index gear 58 and a second index gear 60 from the second gear mechanism 22 provided on the other side of the gear chassis 20. The main drive gear 48 and gear chassis 20 are omitted from FIG. 4, so the strip chassis 12 is visible behind the second gear mechanism 22.

FIGS. 5 and 6 show the opposite side of the strip chassis 12. A first stem or index hub 59 extends from the first index gear 58 and passes through the strip chassis 12 as shown. The first index hub 59 comprises a plurality of compartments 62 configured to receive the blister pockets 8. The compartments are defined by a plurality of upstanding walls 64. Each of the walls comprises a V-shaped formation. A similar index hub 61 is provided on the second index gear 60.

First and second movable peel beaks 66,68 are mounted around the first index hub 59 and the second index hub 61, respectively (see FIG. 17). The peel beaks 66,68 are configured to control and/or advance the peeling of the lidding sheet 10 from the blister strips 4. Operation of the peel beaks 66,68 will be described in detail later.

FIG. 7 shows the chassis cover 16 and first gear mechanism 18 assembled onto the strip chassis 12. First and second index output gears 70,72 are coaxially mounted to the first index hub 59 and the second index hub 61 respectively to transfer rotational drive from the first and second index hubs 59,61 to the remainder of the first gear mechanism 18.

The index output gears 70,72 mesh with respective first and second base drive gears 74,76 and first and second lid drive gears 78,80. The base drive gears 74,76 are connected to first and second spindles 75A and 75B (see FIG. 5) configured to spool up the used base sheet 6 of each blister strip 4. First and second lid take-up drums 81A,81B, configured to spool up the used lidding sheet 10 once it has been separated from the base sheet 6 of each blister strip, are mounted to the lid drive gears 78,80. Each take up drum 81A,81B is mounted to its respective drive gear 78,80 via a torsion spring 83A,83B. The take up drums 81A,81B can thus rotate relative to their drive gears 78,80 against the biasing force of the springs 83A,83B. It will be appreciated that any suitable resilient biasing means may be provided to allow resilient movement between the lid drive gears 78,80 and the take up drums 81A,81B.

It can therefore be seen that separation of the lidding sheet 10 from the base 6 to release a dose of medicament, and individual take-up of the lidding sheet 10 and the base sheet 6, are performed by a single rotation of the main drive gear 54. Therefore, the dispensing cycle of the inhaler is operated solely by movement of the cover 36. The exact performance of the actuation cycle will not be described here for the sake of brevity. Details of the actuation cycle are described in WO 2020/025977, which in incorporated herein by reference.

### Peel Beak

The peel beak gear mechanism 82 is described in closer detail with reference to FIGS. 8-35.

A first embodiment of the pawl 46 is shown in closer detail in FIGS. 8-10. The pawl 46 is mounted on the main drive gear 48. The pawl comprises a plurality of arms 50. The arms 50 are mounted to a hub portion 84. The arms 50 are arranged circumferentially about the hub portion 84. A first curved portion 86 of each arm 50 extends from the hub 84. A second, linear portion 88 extends from each curved portion 86. The arms 50 are therefore substantially J-shaped or hook shaped. The width of the arms 50 tapers or decreases away from the hub portion 84. The linear portion 88 is therefore bendable. As such, an end 90 of each arm 50 is movable in the radial direction (i.e. toward/away from the hub 84). Three arms 50 are provided in the present embodiment, however, it can be appreciated any number of arms may be provided, as required.

The pawl 46 is received within a recess 92 in the body of the main drive gear 48. The pawl 46 is therefore co-planer/concentric with the main drive gear 48. The pawl 46 may be contained within the profile of the main drive gear (see FIG. 15). The recess 92 is substantially circular. The recess 92 comprises a plurality of engagement formations 52. The engagement formations 52 each comprise a sawtooth formation. The sawtooth formation is asymmetric. The ends 90 of the arms 50 are therefore able to pass over the formations 52 whilst travelling in a first direction (due to flexion of the arm 50) and abut against the formation 52 when travelling in a second direction. Whilst the engagement formations comprise inwardly facing protrusions, in other embodiments, they may form recesses, ridges grooves, ribs or other such features.

In the present embodiment, the number of formations 52 is equal to the number of arms 50 (i.e. three). In some embodiments, the number of formations 52 is greater than the number of arms 50 (e.g. multiples thereof). This reduces the rotational lag when rotating in the driving direction. The formations 52 are arranged such that the arms engage the respective formations 52 simultaneously. In other embodiments, not all of the arms (e.g. only a single arm) engage formations 52 simultaneously.

As best shown in FIGS. 9 and 10, the pawl 46 comprises an attachment formation 94 to allow attachment of the of the pawl 46 to the cover 36. The attachment formation comprises a clip or latch 96. The latch 96 is configured to engage an aperture 98 in the cover 36 (see FIG. 1A) and connect thereto. The attachment formation 94 comprises a plurality of raised protrusions 100 configured to be interposed between a plurality of flanges 102 mounted within the cover aperture 98 (see FIG. 1B). The attachment formation thus forms an interlocking arrangement with the cover 36. Therefore, as the cover 36 rotates, the pawl 46 rotates therewith. The protrusions 100 and/or latches 96 extend from the hub 84 of the pawl 46. The protrusions 100 and/or latches 96 are circumferentially spaced about hub 84.

Three latches 96 and protrusions 100 are shown in the present embodiments, however, again, it can be appreciated it can be appreciated any number may be provided, as required. For example, a single latch 96 and/or protrusion 100 may be provided. In alternative embodiments, the latches 96 and/or protrusions 100 may be provided on the cover 36 and engage the pawl 46 accordingly.

A second embodiment of the pawl 46′ is shown in FIGS. 11-14. The alternative pawl 46′ is fixed to, or forms an integral part of, an alternative main drive gear 48′. The ratcheting effect is provided directly between the mouthpiece cover 36′ and the main drive gear 48′ in this embodiment. The alternative pawl 46′ again comprises a plurality of arms 50′, which are configured to engage respective engagement formations 52′. In this arrangement, the formations 52′ are formed as recesses on the inside of the mouthpiece cover 36′ (See FIG. 14). The limbs or arms 50′ are resilient, thus allowing the arms 50′ to pass over the engagement formations 52′.

The arms 50′ are arranged in a V-shape. The apex of the V-shape provides a free end of the pawl 46′. The pawl 46′ is thus tapered. The free-end comprises a flat/blunted end. The arms 50′/pawl 46′ thus define a trapezoidal shape. The arms 50′ define a hollow interior/recess/aperture. The arms 50′ provide a pair of spaced beams. The beams engage at single point (e.g. at the end thereof). The interior at the fixed end of the pawl is rounded. The hollow interior is thus tear-drop shaped.

During inward deflection of the pawl 46′, the radially outer arm 50a′ will be under tension. Conversely, the radially inner arm 50b′ will be under compression. Both of the arms 50a′ and 50b′ may experience at least some bending deformation. The pawl 46′ thus comprise a first beam configured to undergo net tension and a second beam configured to undergo net compression during deflection thereof. The inventor has found that creep deformation and/or fatigue in polymers has a greater severity during tension. Arranging at least one of the beams to placed under compression during deflection reduces the overall creep/fatigue during deflection of the pawl 46′, compared to, for example, a pawl using a single arm. This increases fatigue/creep resistance of the pawl 46′, thereby increasing reliability thereof.

The mouthpiece cover 36′ of the second embodiment comprises a recess 51 configured to receive the pawl 46′. The outer edges of the pawl 46′ thus engage the outer edges of the recess 51. A recess 53 is also provided adjacent each arm 50′ in the pawl 46′ to allow flexion of the arms 50′. It will be understood that the alternative main drive gear 48′ with the integral pawl 46′ could take the place of the engaged drive gear 48 and pawl 46 in the mechanism illustrated in FIGS. 8 and 9.

It should be noted that the second or rear housing portion 26B′ shown in FIG. 2A has a considerably larger aperture 40′, to receive the revised drive gear 48′ and allow the recess 51 of the alternative mouthpiece cover 36′ to interact directly with the pawls 46′.

Referring back to FIGS. 8 and 9, the first drive gear 54 comprises a first plurality of teeth 104 configured to engage and mesh with the main drive gear 48. The first drive gear 54 also comprises a second plurality of teeth 106 mounted coaxially with the first plurality of teeth 104. The second plurality of teeth 106 are configured to mesh with a first plurality of teeth 108 on the second drive gear 56. The first and second plurality of teeth 104, 106 are a continuous about the circumference of the first drive gear 54. Similarly, the first plurality of teeth 108 on the second drive gear 56 are continuous about the second drive gear 56. Both the first and second drive gears 54,56 are thus rotated whenever the main drive gear 48 rotates.

As best seen in FIGS. 15-18, the first drive gear 54 comprises a third plurality of teeth 110. The third plurality of teeth 110 are configured to engage a first peel beak gear 112 and the first index gear 58. The second drive gear 56 comprises a second plurality of teeth 114 configured to engage a second peel beak gear 116 and the second index gear 60.

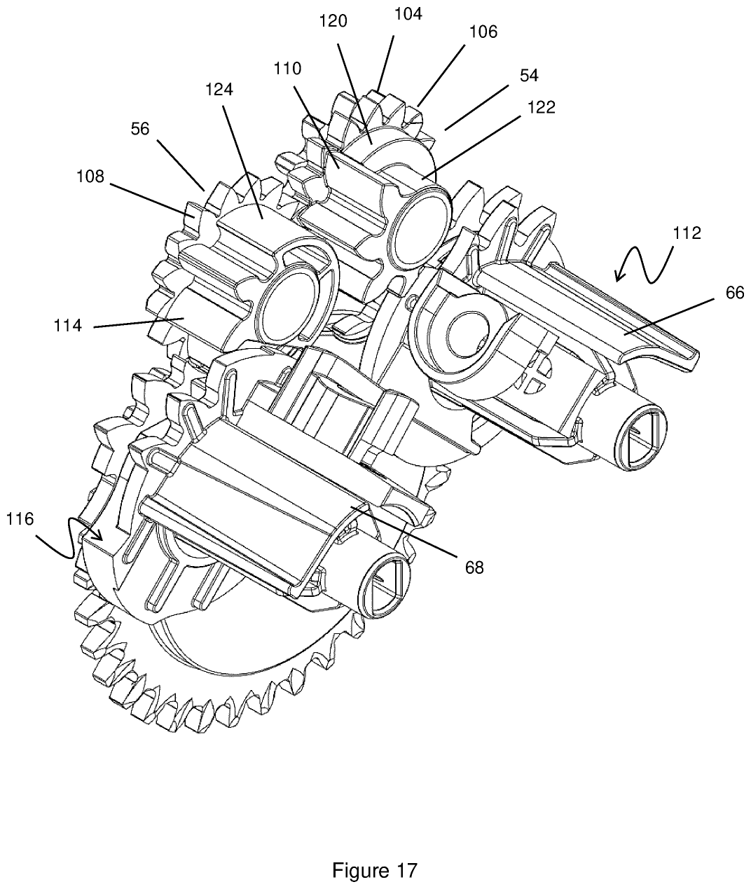

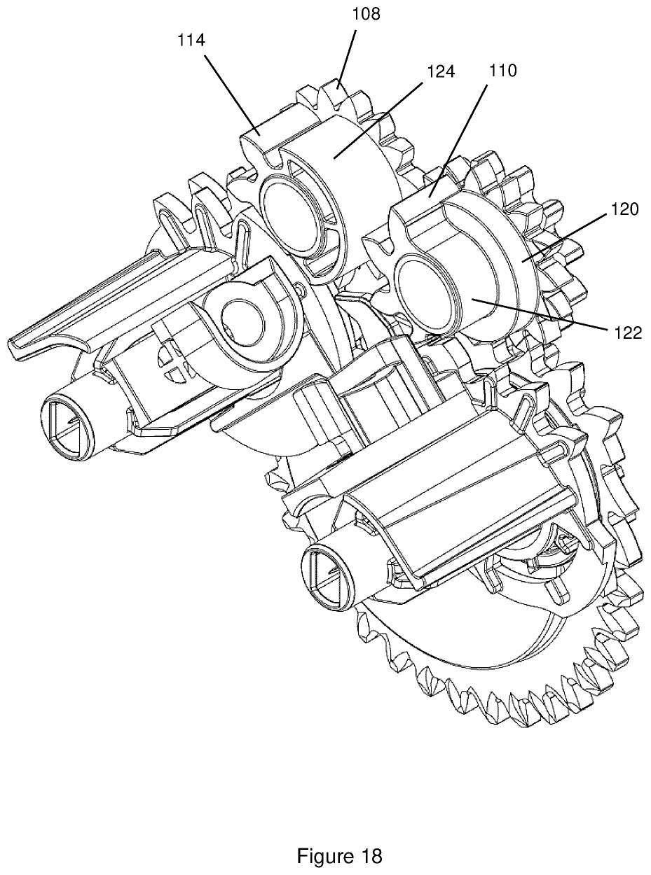

The third plurality of teeth 110 on the first drive gear 54 are discontinuous about the circumference thereof. The third plurality of teeth 110 therefore provide a dwell region 118 where the first drive gear 54 does not mesh with the first index gear 58 or the first peel beak gear 112. As can be seen most clearly in FIGS. 17 and 18, the dwell region 118 comprises a first dwell portion 120 extending for only a part of the width of the third plurality of teeth 110, and a second dwell portion 122, radially inward relative to the first dwell portion 120, extending for the remainder of the width of the third plurality of teeth 110. The first dwell portion 120 aligns with and overlies only the first index gear 58, while the second dwell portion 122 overlies the first peek beak gear 112.

The second plurality of teeth 114 on the second drive gear 56 are also discontinuous about the circumference thereof to provide a dwell region 124. This dwell region 124 extends for substantially the full width of the second plurality of teeth 114, and overlies both the second index gear 60 and the second peel beak gear 116.

The first index gear 58 is shown in FIG. 19A. The first index gear 58 comprises a dwell tooth 126. The dwell tooth 126 allows the first index gear 58 to dwell during rotation of the first drive gear 54 (i.e. relative to the first drive gear 54). The dwell tooth 126 comprises a recess 128. The recess 128 comprises a step or platform. The dwell tooth 126 is thus “L-shaped” or “seat shaped”. In use, the dwell tooth 126 may be driven by a gear tooth engaging a first face 129A and/or by a gear tooth engaging a second partial gear face 129B. In some embodiments, the first face 129A may engage a locking surface. This prevents backwards rotation of the first gear 58 (i.e. locking of the first drive gear 58). The locking surface may be a circumferential surface of another wheel, allowing that wheel to rotate without releasing first gear 58 (i.e. a Geneva-style lock). The first index gear 58 may comprise and/or engage a ratchet mechanism. For example, an engagement member (i.e. a pawl) may engage the first face 129A.

An adjacent tooth 130 comprises an acute cutaway/groove 132 therein. The cutaway 132 allows the third plurality of teeth 110 of the first drive gear 54 to pass over the adjacent tooth 130 to drive the gear face 129B without engaging the adjacent tooth 130. The cutaway 132 is arcuate. A conventional tooth is provided adjacent the cutaway tooth 130. This arrangement is repeated about the circumference of the first index gear 58. In the present embodiment, the arrangement is repeated four times (i.e. spaced by 90 degrees).

The second index gear 60 is shown in FIG. 19B. The second index gear 60 is substantially the same as the first index gear 60, but provided in a mirrored arrangement.

A second embodiment of the index gears 58′,60′ is shown in FIGS. 20A and 20B. The first index gear 58′ comprises a dwell tooth 126′ comprising a step/platform 128′. It can be seen that the step is increased in length relative to the first embodiment. The recess 132′ on the adjacent tooth 130′ is spaced from the dwell tooth 126′ via a channel 133. The channel 133 is U-shaped. This may provide a smooth load profile during operation of the device 2′. The adjacent tooth 130′ comprises a cutaway 132′. The cutaway 132 therefore extends only part way along the radial length of the tooth 130 (i.e. the cutaway 132′ is spaced from the gulley between the tooth 130′ and the dwell tooth 126′). The cutaway 132′ intersects less of the body of the gear tooth 130′ than the cutaway 132 in the gear tooth 130 of the first embodiment of index gear 58. This provides a strong gear tooth 130′.

The peel beak arrangement is shown in detail in FIGS. 21-28. FIG. 21A shows a first embodiment of a first peel beak 66, which is mounted to the first peel beak gear 112. The first peel beak 66 is substantially planar and extends perpendicular to the first peel beak gear 112. The first peel beak 66 curves toward a forward edge 136 thereof. The forward edge 136 provides a peel front for the lidding sheet 10 in use. A rear edge 138 comprises an upstanding ridge 140, which may increase structural rigidity of the first peel beak 66.

The first peel beak gear 112 comprises an aperture 142 therein to receive the stem 59 of the first index gear 58 (see FIG. 24). The peel beak gear 112 is loosely mounted to the stem 59 thereby allowing free movement (i.e. rotation) relative thereto. The peel beak 58 is thus movable independently of the first index gear 58.

The peel gear 112 comprises a bridging element 144. The bridging element 144 is configured to be interposed between the fixed part of the airway manifold 30 and the blister strip 4 in use (best seen in FIGS. 5 and 6) when a dose is presented for inhalation. The bridging element 144 therefore bridges the gap between the blister strip 4 and the fixed part of the manifold 30, and can be considered an extension of the airway. The bridging element 144 comprises a foot portion 146 configured to engage or lie close to the blister strip 4 in use. The foot portion 146 is provided adjacent the aperture to receive the stem 59 of the first index gear 58 to provide a close seal with the blister strip 4. The foot portion 146 extends perpendicularly away from the peel gear 112. The foot portion 146 is provided adjacent the peel beak 66, but the foot portion 146 and a leading edge 136 of peel beak 66 are spaced by a gap 148 to allow the lidding sheet 10 to pass therethrough.

The inner surface 150 of the foot 146 is curved to match the curvature of the aperture 142. The curved surface provides a continuation of the curved forward edge 136 of the peel beak 58. Thus, the foot portion 146 and the peel beak 66 provide a collectively define a curved surface.

As shown in FIG. 21B, the bridging element 144 comprises a sealing element 152 configured to engage the fixed part of the manifold 30 in use. The sealing element 152 comprises an upstanding wall 154 configured to engage the manifold and provide a seal therebetween. The upstanding wall 154 is substantially U-shaped in profile. The upstanding wall 154 may therefore provide a cuff or collar, allowing the sealing element 152 to move in and out of engagement with the manifold 30, as will be described later.

A channel 156 is provided within the upstanding wall 154, and a bore 158 extends through the channel 156. A first aperture 160 is provided in the foot portion 146 and is fluidly connected to the bore 158. The first aperture 160 comprises a grating 162. A second aperture 164 is also provided in the foot portion 146, adjacent the first aperture 160. The second aperture 164 also comprises a grating 166. The second aperture 164 is fluidly connected to the ambient environment. Thus, in use, inhaled air passes through the second aperture 164 and into an opened blister pocket 8. The airflow then entrains the medicament therein and passes out of the blister pocket 8 via the first aperture 160. The entrained medicament then passes through the bore 158 and into the manifold 30. The air then passes though the mouthpiece 34 to the patient.

A second embodiment of the peel beak is shown in FIGS. 22A and 22B. The cuff 154′ of the second embodiment comprises additional flanges 155. The flanges 155 project radially outward from the cuff 154′ and may help to prevent any undesirable deformation of the cuff 154. The second aperture 164′ in the second embodiment additionally comprises a ramped intake. This ensures adequate airflow into the aperture 164′. The ramped intake is provided behind the sealing element 152. The second aperture 164′ in this embodiment also comprises an arched shape rather than a simple round shape as in the previous embodiment.

In FIG. 21A an interlock element 168 is shown mounted to the peel beak gear 112. The interlock element 168 is shown in isolation in FIG. 23A. The interlock element 168 comprises a mounting portion 170 configured to engage the peel beak gear 112. The mounting portion 170 comprises a ring/annulus and is mounted to a rim 172 provided provide on the peel beak gear 112 (see FIG. 23B). The rim 172 engages an inner side of the mounting portion 170. The rim 172 is provided on an opposing side of the peel beak gear 112 to the peek beak 66. The rim 172 surrounds the aperture 142 for the stem 59 of the index gear 58. The rim 172 may only extending partially about the aperture 142. In the present embodiment, the rim 172 comprises a recess 174 therein. The mounting portion 170 comprises a plurality of spacers 176 to ensure the correct position of the interlock 168 on the rim 172 and/or reduce friction.

The interlock element 168 comprises a lever portion 178. The lever portion 178 is configured to engage the second peel beak gear 180, which will be described in detail later. The lever portion 278 extends outwardly from the mounting portion 170 (i.e. radially therefrom). The lever portion 178 comprises a first portion 182 extending from the mounting portion 170 and a second portion 184 extending from the first portion 182. The first portion 182 is trapezoidal and the second portion 184 is triangular. The second portion 184 engages the peel beak gear 112 in use and is angled relative to the first portion 182. The lever portion 178 is therefore angled (i.e. provides an eagle-beak like arrangement).

The interlock element 168 is rotatable relative to the peel beak gear 112. The interlock element 168 rotates about the rim 172. The interlock element 168 is therefore loosely held on the peel beak gear 112 to provide free rotation thereof. The interlock element 168 and/or the peel beak gear 112/rim 172 may comprise a low friction material.

The degree of relative rotation between the interlock element 168 and the peel beak gear 112 is limited. The interlock element 168 comprises a stop member 186 configured to engage the peel beak gear 112 to prevent rotation thereof. The stop member 186 is protrudes from the mounting portion 170 of the interlock 168 and projects out of the plane of the mounting portion 170. The stop member 168 is received within an elongate channel 188 in the peel beak gear 112. Movement of the interlock element 168 is therefore limited by abutment of the stop member 186 against either end 190A, B of the channel 188. The channel 188 is arcuate. The channel 188 thus follows movement of the stop member 186 as the interlock element 168 rotates. In the present embodiment, the channel 188 has a central angle (angular distance) of approximately 80 degrees.

The assembly of the peak beak gear 112 with the index gear 58 and stem 59 is shown in FIG. 24, and an exploded view is shown in FIG. 25.

The walls 64 of the index hub or stem 59 provide a close fit with the aperture 142 of the peel beak gear 112. The walls 64 thus provide a close fit with the peel beak 66. A portion 64A at the base of the walls 64 provides a close fit with aperture 142, preventing undesired movement relative to the peel beak gear 112. The portion 64A comprises a smooth and/or low-friction running surface.

The stem 59 extends from the face of the first index gear 58. The stem 59 is fixed to the first index gears 58 and therefore rotates therewith. The walls 64 are mounted to the stem 59 to define the compartment 62 for individual medicament blister pockets. The stem 59 comprises a recess 135 configured to receive a pin 135A on the first output gear 70 (see FIG. 2). The recess 135 and pin 135A are shaped to prevent relative rotation therebetween.

In an alternative embodiment shown in FIGS. 26-28, the peel beak gear 112′ comprises a plurality of upstanding projections 192 configured to surround the stem 134 of the index gear 58′. The projections are mounted about the circumference of the aperture 142′. An inner surface 194 of the projections comprises a curved surface. The inner surface thus provides a close fit with the stem 134. The projections comprise upstanding (i.e. radially extending) walls 196 to define respective compartments 198. The walls 196 provide strength and dimensional stability. The upstanding walls 196 extending from the peel beak gear 112′ define a V-shape.

The projections 192 extend only partially along the length of the stem 134. The upstanding walls 64 then extend along the remaining portion of the stem 134. The walls 64 on the stem 134 define the compartment 62′ for a blister pocket in this embodiment. The stem walls 64′ in this embodiment define a T-shape.

The close fit of the surface 194 of the projections 192 (i.e. the surface 194 thereof) prevents and/or limits deflection of the stem 134 and/or relative movement (e.g. translational) between the stem 134 and the peel beak gear 112′. This helps to prevent the stem 134 from engaging the peel beak 66, avoiding unnecessary friction within the mechanism and/or translating/bending relative to the foot portion 146, ensuring that the blister pocket is held in the necessary position for effective and repeatable operation.

The interlock 168 is not shown in this embodiment. It should be understood that the interlock 168 is therefore optional, and operation of the device may be achieved without the interlock 168.

Referring back to FIG. 15, the peel beak mechanism is divided into a number of layers:
Layer 1 (L1) comprises main drive gear 48 and the engaged first plurality of teeth 104 on the first drive gear 54.
Layer 2 (L2) includes the second plurality of teeth 106 on the first drive gear 54. The teeth mesh with the first plurality of teeth 108 on the second drive gear 56 (not shown).
Layer 3 (L3) shows the third plurality of teeth 110 on the first drive gear 54, the first dwell portion 120 and the first index gear 58. The second index gear 60 is obscured behind the first index gear 58.
Layer 4 (L4) includes the third plurality of teeth 110, the second dwell portion 122 and the optional interlock element 168.
Layer 5 (L5) shows the third plurality of teeth 110, the second dwell portion 122 and the first peel beak gear 112. The second peel beak gear 116 is obscured behind the first peel beak gear 112.

Operation of the peel beak mechanism will now be described with reference to FIGS. 29-34. The “A” figures show layer 3, the “B” figures show layer 4 and the “C” figures show layer 5 respectively.

The peel beak mechanism is shown in a starting position (i.e. at the start/end of a dispensing cycle) in FIGS. 29A-29C:
In layer 3 (FIG. 29A), the first drive gear 54 engages the dwell tooth 126 on the first index gear 58. The first index gear 58 is locked against backwards (anti-clockwise) rotation by the engagement. Similarly, the second drive gear 56 engages the dwell tooth 126 on the second index gear 60. The first of the teeth 114 on the second drive gear 56 engages the partial gear face 129B on the dwell tooth 126 on the second index gear 60, ready to drive it in the forwards (anti-clockwise) direction.
In layer 4 (FIG. 29B), the teeth 114 of the second drive gear 56 engage a partial gear face 199 (e.g. a step) on the second peel beak gear 116. The second drive gear 56 is primed to drive the second peel beak gear 116 in the forwards (anti-clockwise) direction. The teeth of the first drive gear 54 remain out of engagement with the first peel beak gear 112. Stop member 186 of the interlock element 168 contacts the end 190B of the channel 188 in the first peel beak gear 112 (visible in FIG. 29C). The interlock element 168 is therefore held in a disengaged position from the second peel beak gear 116.
In layer 5 (FIG. 29C), a second cutaway portion 202 is provided on the second drive gear 56. The second cutaway portion 202 extends about a portion of the teeth 114 (e.g. a single tooth or approximately a third thereof). Both the first drive gear 54 and second drive gear 56 are out of engagement with the respective peel beak gears 112,116.

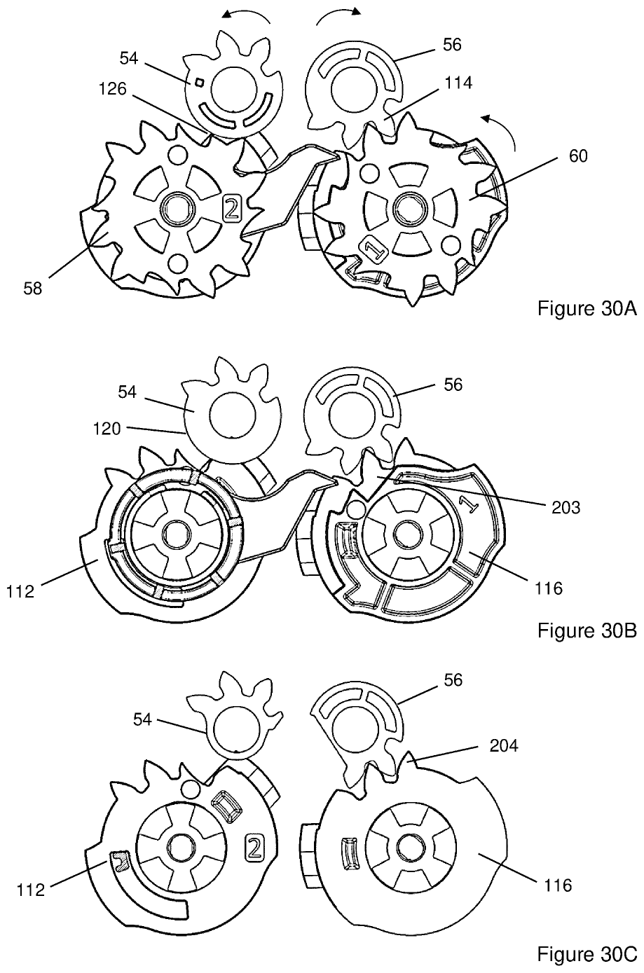

In the next stage, the cover 36 is rotated to rotate the main drive gear 48, which in turn rotates the first drive gear 54 and second drive gear 56. As can be seen in FIGS. 30A-30C, due to the meshing of the first drive gear 54 and second drive gear 56, the gears 54,56 rotate in opposing directions (first drive gear 54 anticlockwise and second drive gear 56 clockwise as shown).
In layer 3, the teeth 114 of second drive gear 56 engage respective teeth of the second index gear 60, thus rotating the second index gear 60 therewith. The first drive gear 54 remains in engagement with the dwell tooth 126, and thus the first index gear 58 remains stationary.
In layer 4, the teeth 114 on the second drive gear disengage from the second peel beak gear 116 due to a cutaway 203 on peel beak gear 116. The interlock element 168 remains held in a position disengaged from the second peel beak gear 116.
In layer 5, the teeth 114 of the second drive gear 56 engage teeth 204 on the second peel beak gear 116. The second peel beak gear 116 thus rotates with the second drive gear 56. The second peel beak rotates away from the airway manifold 30 and mouthpiece 32. The first dwell portion 120 of the first drive gear 54 faces the first peak beak gear 112. The first peel beak gear 112 thus remains stationary. Simultaneous rotation of the second drive gear 56 and the second peel beak gear 116 is provided by engagement of the respective gear faces in layer 4, bringing the teeth 114 of the second drive gear 56 into engagement with the gear teeth on the second peel beak gear 116.

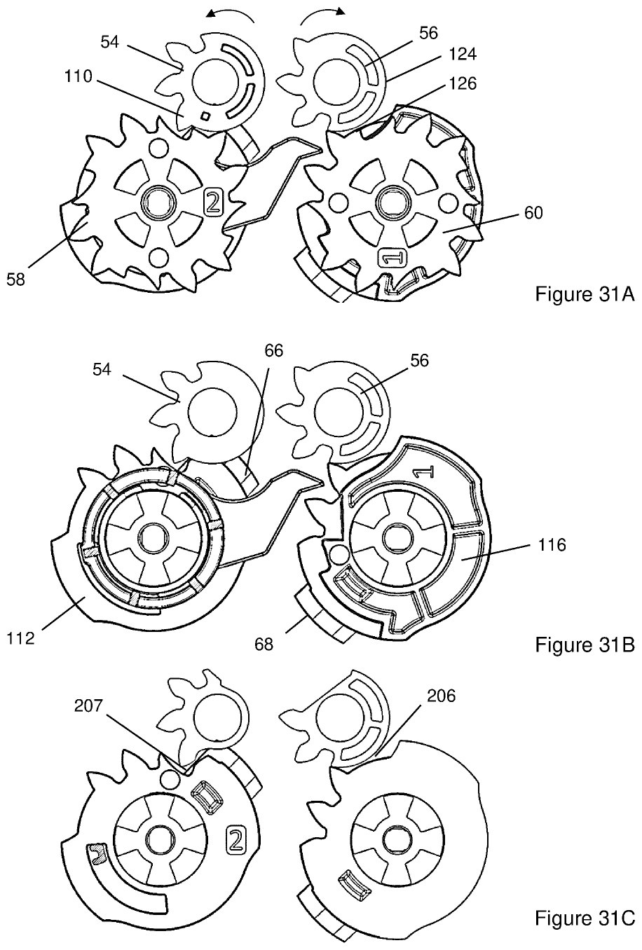

Referring now to FIGS. 31A-31C, with further opening of the cover 36:
In layer 3, the second drive gear 56 has been rotated, rotating the second index gear 60 until the teeth of drive gear 56 disengage from the teeth of the second index gear 60. The first dwell portion 124 is shown engaged with the dwell tooth 126 on the second index gear 60. The second index gear 60 will therefore remain stationary during further rotation of the drive gear 56, and is locked against backwards (clockwise) rotation. The first index gear 58 remains dwelled. The first gear tooth 110 of the first drive gear 54 is now positioned to drive the partial gear face 129B of the first index gear 58, such that further rotation of the first drive gear 54 will effect rotation of the first index gear 58.
In layer 4, the second drive gear 56 and the second peel beak gear 116 remain disengaged from each other. The interlock element 168 remains held in a position in which it is disengaged from the second peel beak gear 116 (by its stop member 186 being in contact with the end 190B of the channel 188 in the first peel beak gear 112).
In layer 5, the second drive gear 56 engages a second dwell portion 206 on the second peel beak gear 116. The second peel beak gear 116 will thus remain stationary or dwelled during further movement of the second drive gear 56. The first peel beak gear 112 remains dwelled between FIGS. 30B and 31B. However, the first gear tooth 114 of the first drive gear 54 is now positioned to drive the partial gear face 207 of the first peel beak gear 112, such that further rotation of the first drive gear 54 will effect rotation of the first peel beak gear 112. The second peel beak 68 is now displaced from the first peel beak 66, having been rotated by approximately 90 degrees.

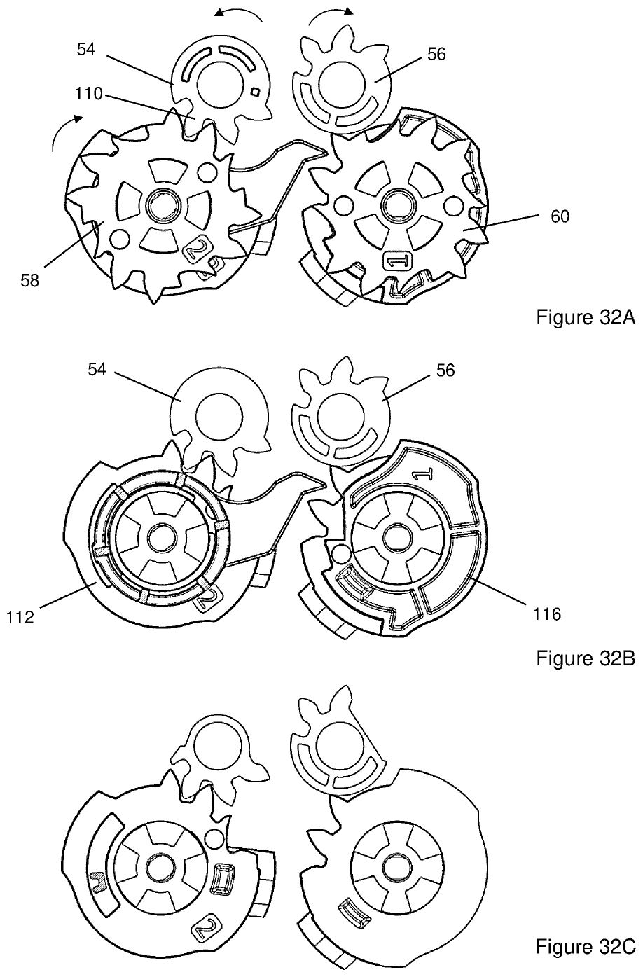

Referring now to FIGS. 32A-32C:
In layer 3, the second index gear 60 remains dwelled relative to the second drive gear 56 as the first drive gear 54 meshes with the first index gear 58, effecting rotation thereof.
In layer 4, the second drive gear 56 and the second peel beak gear 116 remain disengaged from each other. Movement of the first peel beak gear 112 (effected in level 5), and particularly the movement of the end 190B of the channel 188, means that the interlock element 168 is no longer held in its original position.
In layer 5, the second peel beak gear 116 similarly remains dwelled during rotation of the second drive gear 56. The first drive gear 54 meshes with the first peel beak gear 112, thereby effecting rotation thereof.

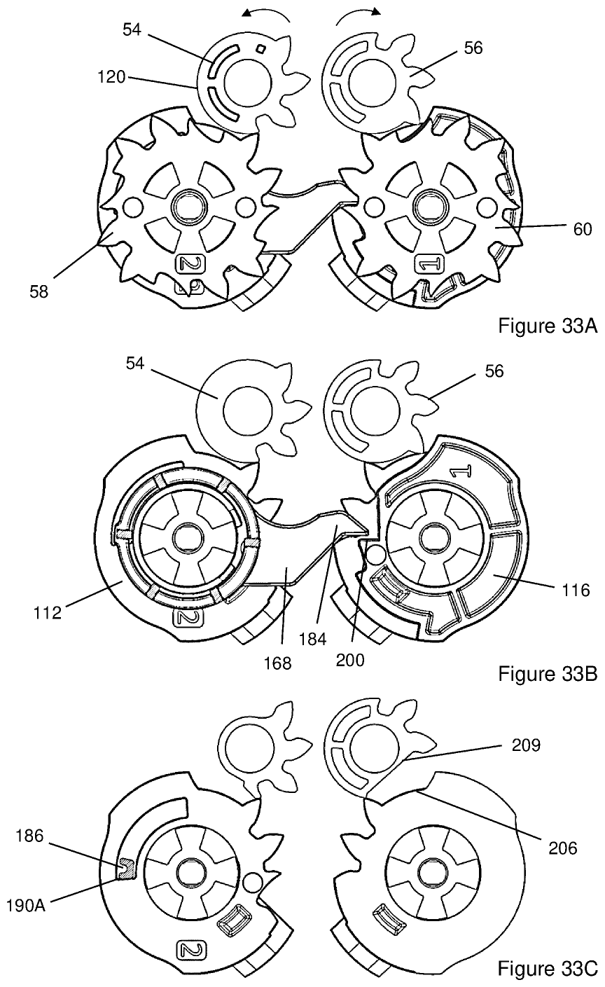

FIGS. 33A-33C illustrate further rotation, to the point where further rotation of the cover 36 has placed the device and mechanism into a primed (i.e. ready for peeling) configuration:
In layer 3, the second index gear 60 remains dwelled during rotation of the second drive gear 56, and the first drive gear 54 has rotated the first index gear 58 to a point where the first dwell portion 120 again engages a dwell tooth 126 of the second index gear 58, so that no further rotational drive is provided. The second index gear 58 is locked against backwards (anti-clockwise) rotation.
In layer 4, the interlock element 168 is moved and held (as described later in layer 5) at a position where its second portion 184 is close to the step 200 on the first peel beak gear 112, such that clockwise rotation of the second peel beak gear 112 will lead to the step 200 coming into contact with the second portion 184. The peel beak gear 116 is therefore prevented from significant clockwise rotation while the interlock element 168 is provided in this position. The second drive gear 56 and the second peel beak gear 116 remain disengaged from each other.
In layer 5, the second peel beak gear 116 remains dwelled during rotation of the second drive gear 56, and a further dwell portion 209 (comprising the tip of the final drive tooth 114) of the first drive gear 54 now also aligns with a second dwell portion 206 on the first peel beak gear 112. It can be seen that the first peel beak gear 112 is now provided in the same position as the second peel beak gear 116 (i.e. in a mirrored position). The peel beak gears 112, 116 are rotated by approximately 90 degrees, and are retained in this position by the engagement of the various dwell portions 209, 124,206. It can be seen that further rotation of the two drive gears 54, 56 will lead to disengagement of the dwell portions 209, 124 from the respective peel beak gears 112, 116. It can also be seen that while the required rotation to effect disengagement is very small in both cases, the required rotation of the second drive gear 56 to disengage the dwell portion 124 from the peel beak gear 116 is smaller than the required rotation of the first drive gear 54 to disengage the dwell portion 209 from the first peel beak gear 112. The stop member 186 of the interlock element 168 engages the end 190A of the channel 188 in the first peel beak gear 112. The interlock element 168 is therefore held in a position such that it prevents the second peel beak gear from rotating substantially in a clockwise direction (shown in layer 4) until the first peel beak gear 112 has rotated in an anti-clockwise direction sufficiently to release the interlock element 168 and thus the second peel beak gear 116. The interlock element 168 helps to fix and coordinate the position and movement of the first and second peel beak gears 112,116 during use, as will be explained further below.

The benefit of providing both layer 4 and layer 5 in the peel beak gear 116 is described with reference to FIG. 29B and FIG. 33C.

FIG. 29B shows layer 4 of the mechanism before the cover 36 (and therefore the mechanism) is operated by the user. The first tooth 114 of the second drive gear 56 engages a partial gear face 199 on the second peel beak gear 116, ready to drive it in the forwards (anti-clockwise) direction when the mechanism is operated. Driving the second peel beak 116 as soon as the cover 36 is operated maximises the angular movement distance of the cover 36 during which work can be put into the system by the user.

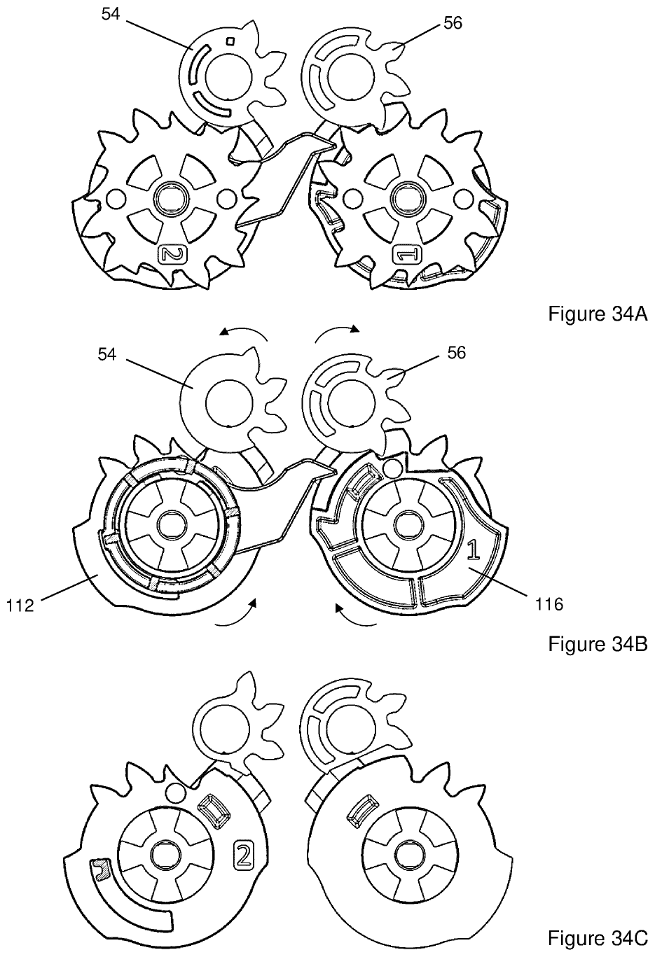

FIG. 33C shows layer 5 of the mechanism just before the final tooth 204 of the second peel beak gear 116 is released by the cylindrical locking face of second drive gear 56. The further dwell portion 209 of the first drive gear 54 retains/abuts a corresponding tooth on the first peel beak gear 112. Once the two peel beak gears are released, they are configured to rotate to the position shown in FIG. 34C.

In layer 4, after completion of the rotation of the second peel beak gear 116 from its position in FIG. 33B to its position in FIG. 34B, the partial gear face 199 is in contact with the first tooth 114 of the second drive gear 56. These features must therefore extend sufficiently from the respective axes of the two components for them to interact.

In layer 5, in order for the second peel beak gear 116 to rotate from its position in FIG. 33C to its position in FIG. 34C, all of the features on peel beak gear 116 (e.g. the teeth 204) do not extend from their respective axes such that they would interact with the release point of the cylindrical locking face of the second drive gear 56. As the rotation of peel beak gear 116 occurs after the release of locking tooth 204 by the release point of the cylindrical locking face of the second drive gear 56, the position of the release point is substantially on the same radius from the axis of the second peel beak gear 116 as the tip of locking tooth 204. Any other features that pass the release point of the cylindrical locking face of the second drive gear 56 during the rotation of the second peel beak gear 116 must therefore be of a lesser extension (i.e. provided radially inward) from the axis of the second peel beak gear 116.

If the second drive gear 56 and the second peel beak gear 116 were provided on the same layer, numerous problems could potentially occur:
1. The tip of the partial gear face 199 of the second peel beak gear 116 would be required to be closer to the axis of the second peel beak gear 116 than the locking tooth 204 (e.g. to avoid interacting with the release point of the cylindrical locking face of the second drive gear 56 during rotation of the second peel beak gear 116).
2. The tip of the first tooth 114 of the second drive gear 56 would be required to be further from the axis of the second peel beak gear 116 than the release point of the cylindrical locking face of the second drive gear 56 (to avoid interacting with the locking tooth 204 during rotation of the second peel beak gear 116).
3. The partial gear face 199 would be required to be further from the axis of the second peel beak gear 116 than the tip of the first tooth 114, in order for the partial gear face 199 to interact with the first tooth 114 at the end of rotation.

Such conditions are incompatible when the second drive gear 56 and the second peel beak gear 116 are provided on the same layer. It can therefore be appreciated that the present arrangement can only be provided when the second drive gear 56 and the second peel beak gear 116 are provided on separate layers. This provides a “drive” layer (layer 4) and a “release” layer (layer 5).

As previously described in relation to FIG. 7, the first and second base drive gears 74,76 are driven directly from the first and second index hubs 59,61, so that tension is applied to a lidding sheet 10 of each blister strip 4 whenever the respective index gear 58,60 is rotated. The lid drive gears 78,80 are driven directly from the first and second index hubs 59,61, such that the base sheet 6 of each blister strip 4 is loosely wound/spooled up when the respective index gear 58,60 is rotated. This serves to take up the lidding sheets 10 and base sheets 6 as doses are advanced and inhaled by a user.

In known systems with a static peel front, the advancing and peeling of a single dose would simultaneously provide an even length of used lidding sheet 10 and base sheet 6, which can be simply wound up by a respective base drive gear and lid drive gear. However, the peel beaks in the present invention provide a peel front which initially moves with the indexed doses. The peel beak gears 112,116 rotate during use, as described above. There is therefore no relative movement of the compartment 62 on the index gear 58 (and thus the base sheet 6) and the leading edge 136 of the peel beak 68. This means that the lidding sheet 10 moves with the base sheet 6 during indexing.

The advancement of a dose thus generates additional slack in the used base sheet 6, which is absorbed by the rotating first and second spindles 75A and 75B driven by the base drive gears 74,76. The advancement of the mechanism also pulls the lidding sheet 10 away from the take up drums 81A,81B and against their rotation by lid drive gears 78,80. Undesirable or unsustainable tension in the lidding foil 10 is avoided by the springs 83A,83B between the lid drive gears 78,80 and the take up drums 81A,81B.

As each dose is indexed and the peel beak gears 112,116 rotate, a corresponding take up drum 81A,81B rotates, against the force of its spring 83A,83B and the rotation of its lid drive gear 78,80, so that used lidding sheet 10 is unwound from the take-up drums 81A,81B. The counter-rotation between the lid drive gears 78,80 and take-up drums 81A,81B is accommodated by the springs 83A,83B, which provide a restorative force to bias each peel beak gear 112,116 toward the “start position” (i.e. biasing the foot portions 146 toward the manifold 30/mouthpiece 32).

In the positions shown in FIGS. 33A-33C, the peel beak gears 112,116 are provided in a “primed position”, with a biasing force applied as described above. The peel beak gears 112,116 move to this primed position asynchronously (i.e. sequentially). However, the asynchronous movement is provided by continuous movement of the cover 36/drive mechanism.

Referring now to FIGS. 34A-34C, the first drive gear 54 and the second drive gear 56 reach their respective starting positions (i.e. a complete a full rotation). With the initial rotation from the positions shown in FIGS. 33A-33C, the second drive gear 56 disengages from the second peel beak gear 116. The second peel beak gear 116 is retained by the second portion 184 of the interlock element 168 acting on the step 200. The first drive gear 54 then disengages from the first peel beak 112. This releases the first peel beak gear 112 and the interlock element 168, which in turn releases the second peel beak gear 116. The peel beak gears 112,116 rotate back to the starting position under action of the biasing means. The device is ready for dispensing of the prepared dose. As the second peel beak gear 116 is released first, and is then held by the interlock element 168 until the first peel beak gear 112 is released, the peel beak gears 112,116 return to their starting positions simultaneously. This may help to accommodate manufacturing tolerances and/or varying use conditions. Because the lid drive gears 78,80 have been actively rotated by the first and second index output gears 70,72 during the indexing, the additional lidding sheet 10 resulting from an opened dose is also wound onto each take up drum 81A,81B.

The dispensing cycle is showed schematically in FIG. 35 with the relative position of each component shown in the graph.
During a first stage 210, the second index gear 60 and second peel beak gear 116 are rotated to the “primed position”. Rotation of the second index gear 60 causes rotation of the index gear stem 134. The upstanding walls 64 on the stem 134 engage the blister pouch, thus advancing a first blister strip (see left hand side of FIG. 6).
During a second stage 212, neither the first index gear 58 nor second index gear 60 are rotated, providing a first dwell stage.
During a third stage 214, the first index gear 58 and the first peel beak gear 112 rotate to the primed position. The upstanding walls 64 on the stem 59,134 engage the blister pouch, thus advancing a second blister strip (see right hand side of FIG. 6).
During a fourth stage 216, neither the first index gear 58 nor second index gear 60 are rotated, providing a second dwell stage.
During a next stage 218, the first drive gear 54 and the second drive gear 56 disengage from the first peel beak gear 112 and the second peel beak gear 116 respectively. The first peel beak gear 112 and the second peel beak gear 116 move under the biasing force of the torsion spring back to their start positions. The progress of the peeling front is controlled by the position of the leading edge 136 of the peel beak 68. The lidding sheet 10 is progressively peeled away from the pockets 8 as the peel fronts move when the peel beak gears 112,116 rotate back to their start positions. The pockets 8 are peeled open and the user may inhale the medicament through the mouthpiece 34. The interlock element 168 ensures the peel beak gears 112,116 move back to the start position synchronously. This ensures that the respective medicaments are dispensed simultaneously.
In the next stage 220, the cover 36 is closed. Due to the action of the ratchet mechanism 46, the main drive gear 48 is not rotated. The drive gears 54,56 remain stationary. The device is ready for the next use.

As shown in the graph in FIG. 35, the first index gear 58 and the second index gear 60 do not rotate back to the starting position. The first index gear 58 and the second index gear 60 are rotated by 90 degrees during each dispensing cycle and rotate in a uni-directional fashion. A similar arrangement applies to the drive gears 54,56, which are rotated by 360 degrees during each dispensing cycle. Thus, movement of the peel beak gears 112,116 and the respective peel beaks 66,68 is at least partially asynchronous from the movement of the drive gears 54,56, index gears 58,60 and the general drive mechanism actuated by the cover 36.

In embodiments where the interlock 168 is not present, it can be appreciated that the synchronous movement of the peel beak gears 112,116 back to the start position may be performed by suitable management of tolerances within the gear train.

The airway manifold 30 and airflow arrangement with the device is described in further detail with reference to FIGS. 36 to 52.

As shown in FIG. 36, the housing 26 comprises an air inlet 222 therein. The air inlet 222 comprises a plurality of elongate apertures. The air inlet 222 therefore comprises a grille or the like, which is covered when the mouthpiece cover 36 is in a closed position, but is open to ambient air when the mouthpiece cover 36 is opened.

The air inlet 222 is configured to overly an inlet 224 in the manifold 30 (see FIG. 37) so that air can pass into the internals of the device. The manifold inlet 224 comprises a plurality of apertures 226. The apertures 226 comprise respective upstanding rims 228. The apertures 226 are elongate and extend toward the mouthpiece 32. The apertures therefore provide a grating like arrangement. It can be appreciated that only a single aperture 226 may provide a sufficient airflow. However, the grating arrangement reduces the ingress of undesirable particulate matter etc.

Referring to FIGS. 38-40, the manifold 30 comprises a branched or Y-shape. The branches may be spaced by approximately 90 degrees. The air inlets 226 are provided on a stem portion 230 of the manifold 30. A pair of retaining flanges 232 are provided for attachment to the chassis of the device. The retaining flanges 232 extend along the stem 230 and extend outwardly therefrom, and are fixed to the chassis in use (see FIG. 37). A second flange 234 surrounds the stem 230 and engages the manifold 30 in use and provides a rim or collar. The second flange 234 helps to secure the manifold 30 to the mouthpiece 32 via a clip, friction fit, or other suitable securing method.

The branch portions comprise respective sealing interfaces 236 configured to engage and seal with the bridging element 144/sealing element 152 on the peel beak gears 112,116. The sealing interface 236 comprises a generally circular opening 238 and an upstanding lip 240 extending partially around the opening 238. A best seen in FIG. 38, the opening comprises a chamber/cavity/channel. The chamber may be substantially circulate/ovate in profile.

The stem 230 and the branch portions are substantially hollow such that the manifold 30 comprises a Y-shaped chamber 239. The chamber 239 is substantially continuous (i.e. provides a single, continuous chamber) so that the stem and both branch portions are fluidly connected. The chamber 239 may provide vorticity, turbidity and/or other non-linear airflow of the air flowing therethrough. This may prevent medicament contacting the side walls of the chamber 239 and/or help to deagglomerate the medicament. The chamber 239 may therefore provide a swirl chamber.

FIGS. 41 and 42 show the manifold 30 in engagement with the first peel beak gear 112. FIG. 43A shows a section through the sealing element 152 in engagement with the lip 240 of the sealing interface 236. The section is taken adjacent the foot portion 146 of the bridging element 144, and shows the lip 240 received within the sealing element 152 of the first peel beak 112. The lip 240 thus plugs or completes the open end of the U-shaped sealing element 152, and provides an abutment to ensure correct alignment of the bridging element 144 and the manifold 30.

FIG. 43B shows a further sectional view of the engagement taken through the cuff 154 of the sealing element 152 and the opening 238 into the manifold 30. The opening 238 is received within the cuff 154 of the sealing element 152 and the effective channel extending between the sealing element 152 and the opening 238 is therefore surrounded on all sides at the interface between sealing element 152 and the opening 238. The sealing element 152 and the sealing interface 236 are at least partially interlocking/inter-engaging (i.e. the sealing element 152 and the sealing interface 236 slide into one another).

It can be seen that a close fit is provided between the between peel beak gear 112,116 and the manifold 30. The peel beak gear 112,116 may engage (i.e. provide contact) with the manifold 30. The peel beak gear 112,116 may seal against the manifold 30. In some embodiments, the peel beak gear 112,116 may not directly contact the manifold 30. However, any space between the peel beak gear 112,116 and the manifold 30 will be designed to avoid any substantial escape of air/medicament. For example, the space will typically be less than 1 mm.

The bridging element 144 rotates with the peel beak gear 112,116. This reduces complexity compared to a design where a linearly sliding or moving airway component is provided, and achieves a similar sealing effect. The use of rotational bearings instead of linear bearings may reduce bearing friction and/or provide a greater control over dimensional tolerances. A closer fit and improved seal can thus be provided with a reduced level of undesirable friction in the system.

Similarly, a close fit is provided between each peel beak gear 112,116 and the compartments 8 of base sheet 6. Any space between the peel beak gear 112,116 and the base sheet 6 is designed to avoid any substantial escape of air/medicament. For example, the space will typically be less than 1 mm. Thus, the bridging element 144 provides a substantially contained/enclosed air pathway between the base sheet 6 and the manifold 30. This, in turn, provides a substantially contained/enclosed air pathway between the base sheet 6 and the mouthpiece 32 to help prevent loss of medicament and/or pressure-difference between the inlet 164 and the outlet 160 of the compartment 8 during use.

A second embodiment of the sealing arrangement, from a peel beak as shown in FIGS. 22A and 22B, is shown in FIGS. 44-45. As best seen in FIG. 44A, the lip 240′ in this embodiment again partially extends around the opening 238 of an alternative manifold 30′ as shown in FIGS. 2A and 2B. However, the lip 240′ in this embodiment further extends tangentially to the opening 238 and thus comprises a substantially linear form. A rim 242 is also provided about the opening 238 in this embodiment. A second rim 243 is spaced from the opening 238 by a recess 244. The recess 244 is configured to receive the cuff 154′ to form a seal, and is curved to allow rotation of the sealing element 152′ of the peel beak 112′ past or along its surface during use. The sealing element 152′ may be correspondingly curved to ensure a close fit. The cuff 154′ is received between the rims 242,243.

FIG. 45A shows a cross-section through a sealing element 152′ of this embodiment, which as before is shaped to provide a close fit with the lip 240′. The alternative sealing element 152′ is similar in many respects to the sealing element 152 previously described.

FIG. 45B shows a cross-section extending through the sealing element 152′ and the sealing interface 236 on the manifold. The cuff 154′ is received within the recess 244 and engages the surface thereof. The cuff 154′ comprises a curved shape configured to provide a close fit with the rim 242 around opening 238. The further flanges 155 extend from the outer surface of the cuff 154′ at the upper/lower edges thereof, which further impede any air passing over/under the flanges 155 respectively. This improves the sealing efficacy of the sealing element 152′, thus preventing undesired air leakage. The air must follow a labyrinthine path to reach the opening 238 from the ambient environment, thus reducing the likelihood of compromising of the pressure-drop generated within the opening 238.

A third embodiment of the manifold 30″ is shown in FIGS. 46 and 47. The manifold 30″ is externally similar to that shown in FIG. 44A, but in this embodiment the chamber 239 comprises a divider 266. The divider 266 at least partially segregates the airflow 268A,268B entering each of the respective openings 238A,238B. This helps to prevent any undesired mixing or agglomeration of the medicaments entrained in the respective airflows 268A,268B and/or helps to ensure adequate aerosolization thereof. It will be appreciated that a dividing central wall or baffle is not necessary to effect differential aerosolisation of two or more separate formulations; asymmetrical features—for example asymmetrical inlet sizes, inlet angles, chamber geometry, etc—can differentially affect the aerosolisation and/or deagglomeration of the formulations without a central wall, or with a shortened or partial central wall.

The divider 266 extends axially along the length of the manifold 30″, between an area adjacent the intersection 270 between the branch portions and an area proximal the mouthpiece end 272 of the manifold 30″. The divider 266 thus extends only partially along the axis of the manifold 30″ and the segregated airflows are free to mix in the area adjacent the mouthpiece end 272. In the present embodiment, the divider 266 extends 80% of the length of the manifold chamber 239, but may extend any length to ensure adequate function thereof. The divider 266 may extend along greater than or equal to 30%; preferably, greater than or equal to 40%; preferably, greater than or equal to 50%; preferably greater than or equal to 60% of the length of the manifold/manifold chamber 239.

The effective size (i.e. cross-sectional area) of the respective segregated chambers 239A,239B is different. This allows for different airflow rates and patterns to be provided within the two chambers 239A,239B and/or different effective inhalation rates for different medicaments through each opening 268A,239B. This in turn allows each chamber 239A,239B to provide the necessary Aerosol Particle Size Distribution (APSD) for a specific medicament. In the present embodiment, this achieved by laterally offsetting the divider 266 from a central axial axis 274 of the manifold chamber 239. This provides a convenient means of providing a consistent overall airflow. However, it can be appreciated that the different airflow rates may be achieved using any suitable means, for example, one or more of: changing the width/thickness of the divider 266; apertures or constrictions in one or both chambers 239A,239B; changing the width of the chambers 239A,239B (i.e. by moving outer walls laterally inward/outwards); providing convolutions or labyrinth pathways; providing bleed pathways/introductory gas pathways; altering the surface finish or geometry of the inner surface of the chambers; or altering the inlet angles.

In some embodiments, the divider 266 is at least partially gas permeable. This provides a degree of segregation but allows some mixing. For example, the divider 266 may be porous, perforated or provided intermittently along the length or across the width of a manifold. In the present embodiment, an axial slot 279 extends partially along the axial length of the divider 266. The slot 279 typically extends less than 10% of the total height of the divider, thus reducing mixing of the airflows 268A,268B. Indeed, where the pressure is approximately balanced on either side of the divider 266, no meaningful mixing takes place as a result of the slot 279. The longer axial slot 279 shown in the figures corresponds to an additional support between parts of a moulding tool used during manufacture of the manifold.

In other embodiments, the divider may be substantially gas impermeable and fully segregate the airflows 268A,268B so that the chamber 239 is fully divided into two fluidly separately chambers 239A,239B.

The size of openings 238A,238B may be adjusted in accordance with the respective size of the chambers 239A,239B. In some embodiments, the respective opening may comprise a consistent size, for example, in embodiments where airflow is primarily limited by the chamber 239 cross-sectional area.

In some embodiments, at least one of the openings 238 and/or the respective chambers 239A,239B may be modified to change the airflow properties therein (e.g. vorticity, dispersion, laminar flow, wall shear stress etc.). The modifications may comprise a different arrangement for each opening 238 or chamber 239A,239B. Such changes in the airflow properties may influence the degree of deaggregation and/or deposition of the dry powder formulation passing therethrough. This may influence the APSD emitted therefrom. The openings 238 and/or chambers 239A,239B may be modified independently from the other, thereby influencing the deaggregation and deposition thereof to suit the particular formulation passing through the respective chamber 239A,239B.

In some examples, the angle of the inlet chamber 238 relative to the axis of the chamber 239 may be varied. This may influence the velocity of the dose inlet flow entering the diffuser chamber 276, and/or the angle at which this flow impinges onto the divider 266. This will influence the impact velocity of the dry powder formulation, which is being carried by the dose inlet flow, onto the divider. The intensity of the resulting particle-to-wall collisions will influence the degree of deaggregation, which will in turn influence the APSD of the drug flow emitted from the inhaler. In the present embodiment, the angle between the dose inlet chamber 238 relative to the axis of the chamber 239 is approximately 45 degrees. The angle between the dose inlet chamber 238 relative to the axis of the chamber 239 may be 20-70; preferably, 30-60 degrees. The angle between the inlet chamber 238 relative to the axis of the chamber 239 may be different for the respective inlet chambers 238A,238B.

In some examples, the openings 238 may open into respective diffusers 276A,276B (i.e. expanding nozzles) within the chamber 239. The diffuser 276A is narrowed compared to diffuser 276B to provide a less diffuse airstream. The diffuser 276A opens into the chamber 239A, resulting in a step 278 provided therebetween. The step may provide a discontinuity in the flow path of the air flowing through the chamber 239. This may allow the cross-sectional area (and thus the velocity of the air flow) in one of the chambers 239A,239B to be increased/decreased without changing the cross-sectional area and air velocity in the other chamber. The position, size, and/or shape (i.e. profile) of the step/discontinuity 278 may therefore be different for each diffuser 276A,B.

It can be appreciated that the change in cross-sectional area at the exit from the diffuser chamber 276 need not be discontinuous (i.e. step-wise). For example, the change in area could be blended in a continuous fashion (i.e. in a ramped arrangement). The magnitude and/or gradient of the ramp may be different between the chambers 239A,239B. In some embodiments, the diffuser 276 and/or step 278 may not be provided.

Such an arrangement may allow an increased volumetric flow of air in the chamber 239, provided by the introduction of the bypass flow (described later). This may allow the chamber 239 to be optimised with partial or complete independence from the deaggregation and deposition mechanisms employed in the diffuser chamber 276. This may also allow the exit from the diffuser chamber 276 to be positioned closer to the centre of the swirling/vortex flow in the chamber 239, where the pressure is lowest. This may be used to maximise the flow rate of air drawn through the opening 238 and diffuser chamber 276 for a given flow-structure in the swirl chamber 239.

It can be appreciated that width and/or axial length of the diffuser of the respective channels 238 may be adjusted as required. In some embodiments, the shape of the respective openings 238 may be different. For example, the cross-sectional shape may be modified to change the vorticity/turbidity of the airflow (e.g. providing a non-circular opening 238). Similarly, the shape, size and/or profile of the chambers 239A,239B may be varied to change the aerodynamic properties thereof. The chambers 239A,239B may be modified independently of each other (i.e. comprise different shapes, sizes and/or profiles).

It can be appreciated that the airflow rate and/or other airflow properties for the respective openings 238 and/or airflows 268A,268B can be adjusted independently of one another, allowing a flexible configuration.

As shown in FIGS. 48 and 49, the manifold inlets 224 are received within the chassis cover 16. The chassis cover comprises respective apertures 280 configured to receive the inlets 224. The apertures 280 are provided within a recessed portion 282 of the chassis cover 16. In full assembly, the first shell portion 26A of the housing overlies the chassis cover 16, with at least a portion of the housing air inlets 222 overlying the recess 268.

The chassis cover 16 comprises a second set of apertures 284 located beneath/adjacent the index drive gears 70,72 when assembled, adjacent the mounting point for the pivoting cover 36. The air inlets 222 the first shell portion 26A also overlie the second set of apertures 284. The second set of apertures 284 comprises an arcuate/curved profile.

Airflow into and out of the device is illustrated in FIGS. 50-52. A first portion of inhaled air 400 enters the inlet 222 in the first shell portion 26A and passes beneath the cover 36, through the housing 26A and into the cavity 286 accommodating the index drive gears 70,72. The air passes through the second apertures 280 and into a cavity 288 beneath the cover chassis 16. The air then passes through the second aperture 164 in the peel bear gear 112 and into the peel blister pocket 8, where it entrains medicament in the unlidded pocket 8. The air exits the pocket 8 through the first aperture 160 (not shown) in the peel beak gear, through the sealing element 152 and into the manifold 30. A similar process is provided for the other medicant. The medicaments may mix in the manifold, depending on the exact configuration of the device.

A second portion of inhaled air 402 enters through the inlets 222 in the housing 26A and then through the inlets 224 in the manifold 30. The air flows into the manifold 30 and mixes with the air 400 comprising the medicament. The second portion of air 402 thus comprises “bypass” air. The mixed air 404 then passes out through the mouthpiece 32 to the user.

In use, the airflow comprising the entrained medicament exiting the diffuser chamber 276 will enter the chamber 239 in a largely axial direction (i.e. as indicted with arrow 404). Conversely, the bypass flow 402 enters in a largely tangential/transverse direction, thus inducing vorticity in the chamber airflow 404. The intensity of the vorticity induced by the bypass flow 402 in the chamber 239 will be influenced by the cross-sectional area of the chamber. Generally, a smaller cross-sectional area will lead to greater vorticity. The shear stresses in the rotating flow will contribute to deaggregation of the medicament, as will the tangential acceleration of the particles as they enter the rotating flow.

In addition, the rotating airflow will tend to induce particle-to-wall collisions between the powder and the surfaces of the chamber 239, further influencing the degree of deaggregation. Increasing the intensity of the swirl will influence each of these deaggregation mechanisms, which in turn will influence the APSD of the drug flow emitted from the inhaler. It will be appreciated that the intensity of the swirl within the swirl chamber 239, and hence the degree of deaggregation, will also be influenced by the location, direction, size and/or geometry of the bypass inlets 224. The present arrangement thus optimises the deaggregation of the powder medicament.

The inventor has found that if the centre of the bypass inlet aperture 224 is moved closer to the central axis of the chamber 239, the intensity of the vorticity of the airflow will generally be reduced (as the inlet airflow 402 is no longer tangential). Thus, the position of the inlet 224 may be adjusted according to the desired vorticity requirements. For example, the bypass inlets 224 may be provided at or adjacent the central axis of the chamber. In such an example, only a single aperture 224 may be provided.

In some embodiments, the bypass flow 402 may enter the chamber 239 in axial direction (i.e. in a direction parallel to the chamber airflow 404). This may reduce or minimise the vorticity of the air within the chamber 239. The bypass flow 402 may enter at an acute angle relative to the axial direction of chamber 239 (i.e. between 0 and 90 degrees). The angle may be chosen accordingly to the desired vorticity level. The inlet 224 may be shaped to guide to the bypass flow at the required angle. For example, the inlets 224 may be curved/angled. In some embodiments, a plurality of inlets 224 may be provided and at least one of the inlets is angled relative to the other to provide axial bypass flow 402.

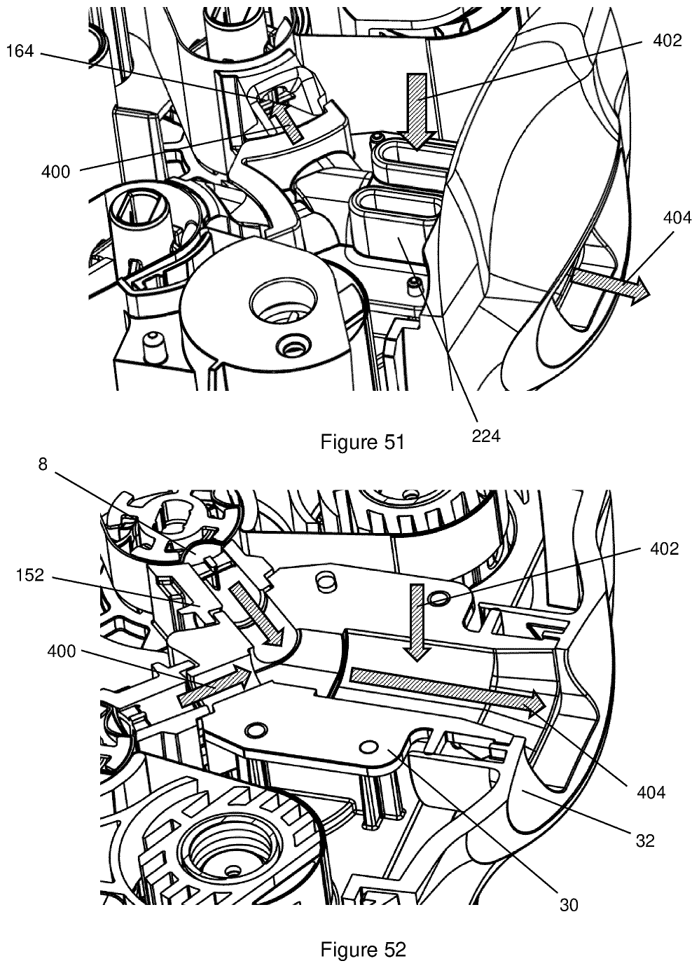

In some embodiments, a plurality of bypass inlets 224 may be provided at one or both lateral sides of the chamber 239. In some embodiments, one or more further inlet may be provided on an opposing side of the chamber 239 to the inlets 224 (i.e. on the underside of the manifold shown in FIGS. 51 and 52). In some embodiments, the inlets 224 may be arrangement circumferentially about the chamber 239. The inlets 224 may be arranged to direct the bypass flow 402 tangentially (e.g. to a provide a cyclonic chamber). The number/size/spacing of the inlets 224 may be adjust according to the desired vorticity.

In some embodiments, one or more of the bypass inlets 224 may be provided in the diffuser chamber 276. In some embodiments, no bypass inlets 224 may be provided.

The connection between the cover and the drive pawl 46 is shown FIGS. 53-56. In a first embodiment, the latches 96 and protrusions 100 described in FIGS. 9 and 10 engage the aperture 98 on one side of the cover 36, as shown in FIG. 53. On the opposing side of the device, shown in FIG. 54, the cover 36 is attached to the device via an attachment mechanism 290. The attachment mechanism 290 comprises a latch 292 configured to engage the cover 36 to prevent removal thereof. The latch 292 snap fits onto the cover 36 and prevents disassembly. A rim/collar 294 comprises an arcuate surface and is configured to engage the inner surface of a second aperture 296 in the cover 36. The attachment mechanism 290 is mounted to the chassis cover 16, and allows rotation of the cover 36.

A second embodiment of the connection is shown in FIGS. 55 and 56. In this embodiment, the cover 36 is held onto the device 36 via a spindle 298. The spindle 298 comprises a journaled shaft 300 and a head portion 302 at the end of the shaft 300. The shaft 300 is received with a collar 304 within the housing 26, which extends laterally across the device between the housing portions 26A,26B. The collar 304 is a part of an alternative strip chassis 12′ (see FIGS. 2A and 2B), and a corresponding chassis cover 16′ and strip chassis 20′ each comprise an aperture to allow the collar 304 to pass therethrough. The collar 304 engages both housing portions 26A,26B and is retained in respective recesses 308 in the housing portions 26A, 26B. A second head 306 is fixed to or formed at the opposite end of the shaft 300 to the first head 302. The second head 306 retains the spindle 298 within the device, thus securing the cover 36 on the device. The collar 304 and/or the spindle 298 may comprise low-friction material (e.g. the collar 304 provides a thrust bearing).

The second head 306 is affixed to or formed on the shaft 300 once the spindle 298 is located within the device. In embodiments where the second head 306 is a separate component, it may be attached via any suitable means, for example, welding adhesive, fasteners, latches etc. In a specific embodiment, the second head 306 is created by deformation of the spindle 298. An end portion 310 of the shaft 300 is heat and deformed (e.g. via heat swaging, ultrasonic welding, or another similar process) to create the head 306. The end portion 310 may be hollow to aid with deformation. In some embodiments, one or both of the heads 302,306 may be detachable from the shaft 300 to allow disassembly of the device.

A first example of the gear chassis 20 is shown in detail in FIG. 57. The gear chassis 20 comprises an anti-back drive mechanism 312 to prevent reverse rotation of the main drive gear 48. The mechanism 312 comprises a set of arms connected at a first end to the gear chassis 20. The opposing second ends of the arms 312 are configured to engage the main drive gear 48. The arms are configured to engage a circumferential rim 314 of the main drive gear 48, illustrated in FIG. 58. The rim 314 is provided by the material of the main drive gear 48 ‘displaced’ to provide the recess 92 to accommodate the pawl 46 on the opposing side of the gear 48. The rim 314 of the main drive gear 48 is rotationally continuous/symmetric, i.e. it does not comprise ratchet features or similar.

The arms 312 extend radially toward the main drive gear 48. Additionally, the arms are angled at least partially tangentially to the rim 314. The arms are resiliently deformable. Therefore, upon rotation in a first direction, the arms 312 flex away from rim 314, thereby reducing/eliminating friction therebetween, and allowing rotation of the main drive gear 48. The arms 312 are therefore biased away from the rim 314. However, upon rotation in a second direction, friction between the arms 312 and the rim 314 applies force down the axis of the arms 312 and causes the arms 312 to flex. The arms 312 are thus biased into engagement with the rim 314. The arms 312 therefore apply increased pressure to the rim 314, thereby increasing the frictional force between the arms 312 and the rim 314, and preventing rotation of the main drive gear 48. The arms 48 are, in their assembled and pre-stressed state, curved to aid with this effect. The reliance on a frictional force acting against a rotationally continuous surface means that the anti-back drive mechanism 312 holds the main drive gear 48 against rotation in any rotational position. There is no need for a ratchet to seat against a particular tooth for rotation to be stopped.

However, it will be appreciated that the frictional force can be increased as necessary by the addition of a rougher surface finish and/or the addition of ratchet steps on the rim 314 and/or the ends of the arms 312. In some embodiments, the rotationally continuous surface of the rim 314 may not be circular in profile; a varying radius may be used to increase or decrease the friction between the rim 314 and the arms 312 at different points in the rotation of the main drive gear 48.

An alternative gear chassis 20′ with a second embodiment of the anti-back drive mechanism 312′ is shown in FIGS. 59 and 60. The mechanism 312′ comprises a set of arms where the first ends of the arms 312′ are connected to the gear chassis 20′ and the opposing ends are configured to engage the main drive gear 48′. The arms 312′ extend out of the plane of the chassis 20′ (i.e. out of the plane of the page). The free ends of the arms are configured to engage ridges 316 on the main gear drive gear 48′. The ridges 316 are provided between pawl arms 50′. The ridges 316 are provided on an opposing side of the main drive gear 48′ to the pawl arms 50′.

The arms 312′ are resiliently deformable, and can therefore snap over the ridges 316 when the main drive gear 48′ is rotated in a first direction. The ends of the arms 312′ abut against the ridges 316 to resist rotation of the main drive gear 48′ in a second direction.

In either of the embodiments, it will be appreciated any number of anti-back drive features 312,312′, (e.g. one or more arms) may be used to achieve the desired effect. The arms may be spaced about the circumference of the main drive gear 48,48′. The arms may be provided in discrete clusters or groups, or may be dispersed evenly about the main drive gear 48,48′.

A further embodiment of the main drive gear 48″ is shown in FIG. 61. The main drive gear 48″ comprises a modified tooth arrangement 318. The section of the teeth indicated from A-A′ will be described. A perfect circle 320 is shown in dashed lines for reference. Starting at A, the pitch line of the teeth 318 steadily increases in radius around the gear 48″ toward A′, so that both the peaks and throughs of subsequent teeth 318 progressively extend further radially from a central point 322 to a local maximum at 324. The radius of the pitch line of the teeth 318 then decreases stepwise to a local minimum at 326. The local minimum 326 is provided radially outward from the starting point A. From the local minimum point 326, the radius of the pitch line of the teeth 318 again increases to a maximum at 328, which has a greater radius than the local maximum at 324. The radius of the pitch line of the teeth 318 then drops stepwise again to return to the starting radius at A′. This arrangement is repeated two more times about the circumference of the main drive gear 48.

The first drive gear 54″ in this variant is configured to correspondingly mesh with the main drive gear 48″. The teeth 330 on the first drive gear 54″ are therefore modified. Starting at B, the teeth 330 have a maximum radius 332 corresponding with the minimum provided at A or A′. The pitch line radius of the teeth 330 then progressively decreases to a local minimum 334, which corresponds to the local maximum radius 324 on the main drive gear 48″. The pitch line of the teeth 300 then increases stepwise to a local maximum radius which corresponds with the local minimum 326 on the main drive gear 48″. The radius then progressively decreases back to a minimum at the starting position at B. This ensures the spacing between the gears 48″,54″ remains constant.

A single rotation of the main drive gear 48″ provides three rotations of the first drive gear 54″. Accordingly, each 120° rotation of the main drive gear 48″, corresponding to the opening of a mouthpiece cover 36, completely rotates the first drive gear 54″.

The varying radius of the pitch line of the teeth 318 on the main drive gear 48″ and the corresponding change for the teeth 330 on the first drive gear 54″ steadily reduces the gear ratio as the effective radius of the main drive gear 48″ reduces relative to the first drive gear 54″ during rotation from the position shown, and increases the ratio during the stepwise pitch change after each minimum 326,A,A′ on the main drive gear 48″. Thus the effective gearing ratio between the main drive gear 48″ and the first drive gear 54″ is variable. This varying ratio helps to smooth varying force profiles and reduce peak forces experienced during rotation of the cover 36.

A schematic force diagram is shown in FIG. 62, with a first trace 338 showing the force required open the cover 36 against the mechanism in the first embodiment during a cap opening sequence. It can be seen that the required force gradually increases during the first half of cap opening and again, to a slightly greater degree, during the second half of the cap opening. The increases are largely due to the tensioning of torsion springs 83A,83B, as described above, as the peel beaks sequentially rotate to their primed positions. A second trace 340 shows the force required during the same dispensing sequence when the main drive gear 48″ of FIG. 61 is incorporated. The internal resistance of the mechanism is substantially unchanged, but the two steady decreases in gear ratio during each 120° of rotation compensate for the two different increasing resistive force profiles. The cover 36 thus requires a substantially constant force across most of the opening operation. The steps up in gear ratio correspond to a small dwell period, provided between movement of the two peel beaks, where the resistance is minimal, and the release at the end of cap opening, where an increased resistance provides useful feedback for a user.

Another alternative first drive gear 54′ and second drive gear 56′ are shown in FIGS. 63 and 64. The gears 54′,56′ do not include the varying radius described above, but the meshing teeth 106′,108′ are optimised for unidirectional rotation because the drive gears 54′,56′ are only driven in single direction.

The teeth 106′, 108′ have a generally triangular shape, but their profile is slightly modified to provide different first and second surfaces 336,338 on opposite sides of each tooth 106′, 108′. A radial reference line 340 is shown between the apex 342 of a tooth 108′ and a rotational centre 344 of the second gear 56′. As shown in FIG. 64, a first angle C is defined between the radial line 340 and the tangent of the first surface 336 and a second angle C′ is defined between the radial line 340 and the tangent of the second surface 338. The angles C and C′ are different, giving each tooth an asymmetrical shape. Each tooth thus generally follows the form of a scalene triangle rather than an isosceles triangle, but the first and/or second surfaces 336,388 have a slight convex curve. The tangent of the surfaces 336,388 may be define by the tangent at the apex of the curved surfaces.

In the present embodiment C=30° and C′=39°. The engagement surfaces 336,338 thus extend a difference of 9° relative to the radial reference line 340. The difference may be greater than or equal to 2°; preferably, greater than or equal to 5°. The difference may be between 2° and 20°; preferably, between 5° and 15°. The angle C may between 30° and 50°; preferably, between 35° and 45°. The angle C′ may between 20° and 40°; preferably, between 25° and 35°.

The teeth 106′ on the first gear 54′ have the same shape and orientation around the first gear 54′. The meshing of the gears 54′,56′ thus means that the first surfaces 336 of the teeth 106′, 108′ face each other, and the second surfaces 338 likewise face each other. The first surfaces 336 engage during rotation, so that drive is transferred between the gears 54′,56′ via the faces with a steeper profile relative to the radius/reference line 340. The shallower angle of the second surfaces 338 at the trailing edges provides additional depth to each tooth, and provides stronger teeth. The steeper profile angle of the contact surfaces 336 allows increased transfer of force from the gear tooth on gear 54′ to the gear tooth on gear 56′, with a reduced resultant force towards the axis of each wheel (increasing the friction at the bearing of each wheel). Additionally, because clearance is only required for the trailing edges 338 of the teeth 106′, 108′, dwells at the start of driving and uncertainty over gear position that would otherwise result from clearance provided at the leading edges of uniform teeth can be minimised or avoided.

Similar tooth design can also be incorporated into the various other gears within the inhaler. The design is possible because the overall mechanism design only requires gears 54′,56′ to be driven in one direction, so symmetrical teeth are not required.

A third embodiment of the inhaler device 2″ is shown FIGS. 65-76. Several aspects of the third embodiment are substantially the same as the first and second embodiments, and like features will not be described again for the sake of brevity.

The third embodiment 2″ essentially provides a simplified version of the mechanism from the first and second embodiments. As best seen in FIGS. 67 and 68, the peel beak gears and the respective peel beaks present in the first and second embodiments are not provided. Thus, the first drive gear 54″ and the second drive gear 56″ only transfer drive from the main drive gear 48″ to the respective index gears 58″,60″. The first drive gear 54″ comprises the first dwell portion 120″ for managing the mechanism, but the second dwell portion 122 as shown, for example, in FIG. 17, is not provided. Driving of the index gears 58″,60″ occurs as previously described (i.e. the movement of the index gears 58″,60″ is asynchronous).

Peeling of a lidding sheet 10 from the blister strip 4 is effected by biasing force of the take up mechanism (i.e. rotation of the respective take-up drums 81A″, 81B″). Thus, each blister strip 4 is peeled simultaneously with the advancement of each index gear 58″,60″, without the use of the peel beaks to control or manage the position of the peel front during the indexing operation.

The first index gear 58″ is shown in isolation in FIG. 69. The upstanding walls 64″ are parallel or near-parallel (e.g. less than 2-3 degrees divergence). As no peel beak is provided, a spacer 346 is provided adjacent the first index gear 58″. This spaces the blister strip 4 from the first index gear 58″.

The main drive gear 48″, shown in FIG. 70, comprises deformable arms 50″ as previously described. The main drive gear 48″ also comprises an aperture 348 for rotationally mounting the main drive gear 48″ to a spindle 350 on the gear chassis 20″ (see FIG. 65). Notches or grooves 352 are provided about the aperture 348 to allow a tab or the like on the spindle 350 to pass through the aperture 348. The tab then retains the main drive gear 48″ on the gear chassis 20″ when the tab and aperture are out of alignment.

The mouthpiece cover 36″ is partly shown in FIG. 71. The mouthpiece cover 36″ comprises a part-circular upstanding wall 51″ with a single notch/groove 52″ configured to engage the end 90″ of a first arm 50″ of the main drive gear 48′″ as the mouthpiece cover 36″ is opened. It can be appreciated that only a single notch or engagement formation 52″ is sufficient for operation of the ratchet mechanism on the main drive gear 48″ to drive the main drive gear 48″.

In use, the upstanding wall 51″ fits within a circular opening in a housing component when the inhaler device is assembled. A cross sectional view of the interface is shown in FIG. 72. An inwardly extending formation 55″, similar in design to engagement formation 52″, is provided on an inner surface of the circular opening to engage with the end 91″ of a different arm 50″ of the main drive gear 48″ and resist back rotation as the mouthpiece cover 36″ is closed and the first deformable arm 50″ is made to deflect to allow the engagement formation 52″ to pass. The gap 53″ provided in the upstanding wall 51″ allows the mouthpiece cover 36″ to rotate relative within the opening in the housing despite the presence of the inwardly extending formation.

An alternative airway manifold 30″ is shown in FIGS. 73-75. As the device 2″ of the third embodiment does not use a peel management mechanism, a sealing element 354 is provided at each inlet to the manifold 30″. The sealing element 354 may abut or be provided close to the blister strip 4 in use. This prevents or limits loss of medicament during inhalation through the manifold 30″. A respective sealing element 354 is provided for each branch of the manifold 30′″.

The sealing element 354 comprises first and second apertures 160″, 164″ similar to those provided in the bridging element 144 previously described in FIG. 21A. The first aperture 160″ is fluidly connected to the interior space of the manifold 30″, and the second aperture 164″ is open to the ambient environment around the manifold 30″. The first and second apertures 160″, 164″ each comprise a cruciform grating/grille 162″, 166″. As previously described, in use, inhaled air passes through the second aperture 164″ into an opened blister pocket 8 and then carries the medicament out of the blister pocket 8 and into the manifold 30″ via the first aperture 160″.

The engagement surface 362 of the sealing element 354 is curved/arcuate. The engagement surface 362 thus follows the curved surface of the blister strip 4 adjacent the manifold 30″.

As best seen in FIG. 74, the sealing element 354 and the manifold body 356 are formed as separate components and joined together during manufacturing. The sealing element 354 comprises a short section of channel 364 configured to be received within a recess or sleeve 366 in the manifold 356. The channel 364 and the sleeve 366 provide a tight fit or otherwise sealingly engage at points around the interface. The channel 364 and the sleeve 366 may comprise an interference fit. Alternatively, the channel 364 may be permanently fixed to the interior of the sleeve 366 via adhesive, welding, fasteners etc. The channel 364 surrounds the first aperture 160″ and thus provides a passageway for the inhaled air and medicament into the manifold body 356. The sealing element 354 extends the effective length of a branched inlet to the manifold cavity. The extended length of the inlet improves the aerodynamic flow into the manifold 30″ (e.g. may produce less turbulence).

The inclusion of the channel 364 adjacent the cruciform grille 162″ and the general complexity of the design of the sealing element 354 and the manifold body 356 make moulding of the entire manifold 30″ as a single piece difficult. This has been addressed by using a three-part manufacture as shown in FIG. 74. For assembly, the manifold body 356 comprises a hollow rod or post 368 configured to be received within a corresponding aperture 370 in the sealing element 354. The post 368 and aperture 370 may form a tight-fitting arrangement, and the end of the post 368 can then be heat staked to secure the components together.

The manifold 30″ of the third embodiment also provides additional bypass channels in the flow path from each blister to the main manifold body 356 during use. Bypass or bleed apertures 358 are formed in each sleeve portion 366 of the main body 356, and provide a flow path for air from the exterior of the manifold 30″ even when a channel 364 of the sealing element 354 is received within the sleeve 366. The bypass apertures 358 allow additional flow from outside the manifold 30″ to separately join the entrained medicament flow from each blister before the separate flows enter the main manifold body 356. The bypass apertures 358 are positioned and shaped so that the additional flow is aligned with the flow of entrained medicament from a blister. This may help to avoid medicament from depositing on the walls of the manifold 30″ during use, improving delivery.

The manifold 30″ also includes a pair of inlets 224″, similar to those seen in earlier embodiments, allowing bypass flow from outside the manifold directly into the main manifold body 356.

A take-up drum 81″ of the third embodiment is shown in detail in FIGS. 76 and 77. The take-up drum comprises a grip member 370 configured to grip or hold the blister strip 4. Thus, as the drums 81″ rotate, a used lidding sheet 10 is wound onto each drum 81″. The grip comprises a slot. The slot is open at one end 372. The grip 370 thus provides a cantilever or flange. The flange 370 is curved to conform to the shape of the take-up drum 81.

A plurality of elongate ridges/grooves 374 are provided on the surface of the take-up drum 81. The ridges 374 are spaced about the circumference of the take-up drum 81. The ridges 374 increase the effective diameter of the take-up drum 81 and/or increase the grip thereof.

1.-31. (canceled)

32. An airway manifold for an inhaler device, the manifold comprising first and second manifold inlets for receiving first and second medicaments, and a single outlet in communication with a mouthpiece;
wherein the manifold provides a first flow path between the first manifold inlet and the single outlet, and a second flow path between the second manifold inlet and the single outlet, and wherein the manifold is designed or configured to provide a difference in flow through the first and second flow paths.

33. An airway manifold according to claim 32, wherein the first and second flow paths are asymmetrical.

34. An airway manifold according to claim 33, wherein the volume of the first flow path is different from the volume of the second flow path.

35. An airway manifold according to claim 33, wherein the internal form of the first flow path is different from the internal form of the second flow path.

36. An airway manifold according to claim 32, wherein the surface finish of the first flow path is different from the surface finish of the second flow path.

37. An airway manifold according to claim 32, wherein the manifold is at least partly split into first and second chambers by a dividing wall, wherein the first chamber provides the first flow path and the second chamber provides the second flow path.

38. An airway manifold according to any of claim 37, wherein three or more chambers are provided.

39. An airway manifold according to claim 37, wherein a separate bypass air inlet is provided into each chamber.

40. An airway manifold according to any of claim 39, wherein the bypass air inlets are asymmetrical.

41. An airway manifold according to claim 32, wherein the first and second manifold inlets are asymmetrical.

42. An airway manifold according to claim 41, wherein the size of the first manifold inlet is different from the size of the second manifold inlet.

43. An airway manifold according to claim 41, wherein the angle of the first manifold inlet is different from the angle of the second manifold inlet.

44. An airway manifold according to claim 41, wherein the surface finish of the first manifold inlet is different from the surface finish of the second manifold inlet.

45. An inhaler device comprising an airway manifold according to claim 32.

46-51. (canceled)
